SEQUENCE LISTING (66 sequences)

SEQ ID NO: 1 - DNA, 839 bp, Pichia pastoris
gataaaaaaaaacgagacgataagatgaggaaggtaccacacatgggcattcttagtgcgcgagagatgattagcatcgagggaaagcttaaacatctttggtctacgtaagcagagaccaggcactagcaagcctaattagggttagggaattgaatgtcagcaaaagctgaggcggcttccgagggccaatagaataagaaagaacaacttagggcgcaaacctgattgcgattttggggctttccttggaaaagacttgatccctacgctgtggaaggcgcactactatcgaagctccctctaacctcccaaaggagaaggaagggaaaaaaaaatagtgacaaaaagaaaacaaagagcccaagacctctatcgccccatcgcccagatctcctatcagcaaaattatgtaagctgcatcttttggtgagctaaaggggactttcgcgctaacaaaaagagcaaacttgtttgttgggtgattgttgggtgttcaaggcacgactttctaatctaccttgcattgacagattcttccaactgcgcccgatataacgtagcattgccaggtaatgatggtatactttacatggtcacactacgacgctcaacatcagtccctcttagtggaaccacaacttgctcgttgaattttggagcgtaatgtgtcatgttgggtcctgcaaaaagaaaagttggatcccataaatttagactttgtaggatgacaatctacagagatttctcgaacttcgggccttcctataaaacaagataaactccttcctctttctctttccttctctttagtcttctcacttcatctacgccacaca

SEQ ID NO: 2 - DNA, 1000 bp, Pichia pastoris
attactgttttgggcaatcctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaa

SEQ ID NO: 3 - DNA, 940 bp, Pichia pastoris
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 4 - DNA, 483 bp, Pichia pastoris
agatcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactat

SEQ ID NO: 5 - protein, 20 aa, Pichia pastoris
MLSTILNIFILLLFIQASLQ

SEQ ID NO: 6 - DNA, 60 bp, Pichia pastoris
atgctatcaactatcttaaatatctttatcctgttgctcttcatacaggcatccctacag

SEQ ID NO: 7 - protein, 31 aa, Pichia pastoris
MLSLKPSWLTLAALMYAMLLVVVPFAKPVRA

SEQ ID NO: 8 - DNA, 93 bp, Pichia pastoris
atgctgtcgttaaaaccatcttggctgactttggcggcattaatgtatgccatgctattggtcgtagtgccatttgctaaacctgttagagct

SEQ ID NO: 9 - protein, 18 aa, Pichia pastoris
MFLKSLLSFASILTLCKA

SEQ ID NO: 10 - DNA, 54 bp, Pichia pastoris
atgttcctcaaaagtctccttagttttgcgtctatcctaacgctttgcaaggcc

SEQ ID NO: 11 - protein, 16 aa, Pichia pastoris
MRIFHWILFFITTSLA

SEQ ID NO: 12 - DNA, 48 bp, Pichia pastoris
atgagaatttttcactggattctcttctttattaccacttcgcttgcc

SEQ ID NO: 13 - protein, 16 aa, Pichia pastoris
MNLYLITLLFASLCSA

SEQ ID NO: 14 - DNA, 48 bp, Pichia pastoris
atgaacttgtacctaattacattactattcgccagtctatgcagcgca

SEQ ID NO: 15 - protein, 18 aa, Pichia pastoris
MSYLKISALLSVLSVALA

SEQ ID NO: 16 - DNA, 54 bp, Pichia pastoris
atgtcttacttgaaaatttccgctttgctttcagttttgtccgtcgccttggcc

SEQ ID NO: 17 - protein, 18 aa, Pichia pastoris
MMYRNLIIATALTCGAYS

SEQ ID NO: 18 - DNA, 54 bp, Pichia pastoris
atgatgtacaggaacttaataattgctactgcccttacttgcggtgcatacagt

SEQ ID NO: 19 - protein, 19 aa, Pichia pastoris
MKISALTACAVTLAGLAIA

SEQ ID NO: 20 - DNA, 57 bp, Pichia pastoris
atgaagatatccgctcttacagcctgcgctgttactctagctggtcttgcaattgca

SEQ ID NO: 21 - protein, 20 aa, Pichia pastoris
MKLSATLLLSVFTSIQSAYA

SEQ ID NO: 22 - DNA, 60 bp, Pichia pastoris
atgaagttatcagcaaccttactgctctccgttttcacttccatccagtctgcctacgct

SEQ ID NO: 23 - protein, 20 aa, Pichia pastoris
MIFNLKTLAAVAISISQVSA

SEQ ID NO: 24 - DNA, 60 bp, Pichia pastoris
atgatctttaatcttaaaacactggctgcggttgcaatctccatttcacaagtgtctgca

SEQ ID NO: 25 - protein, 20 aa, Pichia pastoris
MSCLSHLIASVCFLLCIVEA

SEQ ID NO: 26 - DNA, 60 bp, Pichia pastoris
atgagttgtttatcccatcttatcgctagcgtatgttttttgttatgcatagtagaagct

SEQ ID NO: 27 - protein, 19 aa, Pichia pastoris
MRTQKIVTVLCLLLNTVLG

SEQ ID NO: 28 - DNA, 57 bp, Pichia pastoris
atgagaacacaaaagatagtaacagtactttgtttgctactaaatactgtgcttgga

SEQ ID NO: 29 - protein, 13 aa, Pichia pastoris
MLIGSCLLSSVLA

SEQ ID NO: 30 - DNA, 39 bp, Pichia pastoris
atgttaataggatcctgcctattgagttcagtcttggca

SEQ ID NO: 31 - protein, 16 aa, Pichia pastoris
MLSILSALTLLGLSCA

SEQ ID NO: 32 - DNA, 48 bp, Pichia pastoris
atgttgtccattttaagtgcattaactctgctgggcctgtcttgtgct

SEQ ID NO: 33 - protein, 18 aa, Pichia pastoris
MQVKSIVNLLLACSLAVA

SEQ ID NO: 34 - DNA, 54 bp, Pichia pastoris
atgcaagttaaatctatcgttaacctactgttggcatgttcgttggccgtggcc

SEQ ID NO: 35 - protein, 23 aa, Pichia pastoris
MSFSSNVPQLFLLLVLLTNIVSG

SEQ ID NO: 36 - DNA, 69 bp, Pichia pastoris
atgtcattctcttccaacgtgccacaacttttcttgttgttggttctgttgaccaatatagtcagtgga

SEQ ID NO: 37 - protein, 23 aa, Pichia pastoris
MKLLNFLLSFVTLFGLLSGSVFA

SEQ ID NO: 38 - DNA, 69 bp, Pichia pastoris
atgaaattgttgaactttctgcttagcttcgtaactctgttcggactattatcaggttctgtgtttgca

SEQ ID NO: 39 - protein, 21 aa, Pichia pastoris
MKFPVPLLFLLQLFFIIATQG

SEQ ID NO: 40 - DNA, 63 bp, Pichia pastoris
atgaaatttcctgtgccacttttgtttctactgcagctgttctttattattgcaacacaagga

SEQ ID NO: 41 - protein, 19 aa, Pichia pastoris
MKFAISTLLIILQAAAVFA

SEQ ID NO: 42 - DNA, 57 bp, Pichia pastoris
atgaagttcgcaatttcaacacttcttattatcctacaggctgccgctgtttttgct

SEQ ID NO: 43 - protein, 20 aa, Pichia pastoris
MKLSTNLILAIAAASAVVSA

SEQ ID NO: 44 - DNA, 60 bp, Pichia pastoris
atgaagctctccaccaatttgattctagctattgcagcagcttccgccgttgtctcagct

SEQ ID NO: 45 - protein, 19 aa, Pichia pastoris
MHPYTVVFARLLLGVFSTA

SEQ ID NO: 46 - DNA, 57 bp, Pichia pastoris
atgcatccatacaccgtagtatttgcgcgcctcctcctgggtgttttctcaactgcc

SEQ ID NO: 47 - protein, 18 aa, Pichia pastoris
MKFFYFAGFISLLQLIFA

SEQ ID NO: 48 - DNA, 54 bp, Pichia pastoris
atgaaatttttttactttgcggggttcatatctctgttacagctgatattcgcc

SEQ ID NO: 49 - protein, 24 aa, Pichia pastoris
MIFDGTTMSIAIGLLSTLGIGAEA

SEQ ID NO: 50 - DNA, 72 bp, Pichia pastoris
atgatatttgacggtactacgatgtcaattgccattggtttgctctctactctaggtattggtgctgaagcc

SEQ ID NO: 51 - protein, 16 aa, Pichia pastoris
MKSQLIFMALASLVAS

SEQ ID NO: 52 - DNA, 48 bp, Pichia pastoris
atgaaatctcaacttatctttatggctcttgcctctctggtggcctcc

SEQ ID NO: 53 - protein, 19 aa, Pichia pastoris
MKLAALSTIALTILPVALA

SEQ ID NO: 54 - DNA, 57 bp, Pichia pastoris
atgaagctcgctgcactctccactattgcattaactattttacccgttgccttggct

SEQ ID NO: 55 - protein, 21 aa, Pichia pastoris
MQFNSVVISQLLLTLASVSMG

SEQ ID NO: 56 - DNA, 63 bp, Pichia pastoris
atgcaattcaacagtgtcgtcatcagccaacttttgctgactctagccagtgtctcaatggga

SEQ ID NO: 57 - protein, 20 aa, Pichia pastoris
MVSLTRLLVTGIATALQVNA

SEQ ID NO: 58 - DNA, 60 bp, Pichia pastoris
atggtttctttaacaagactactagttaccggaatcgccaccgctttgcaggtgaatgcc

SEQ ID NO: 59 - protein, 19 aa, Pichia pastoris
MSTLTLLAVLLSLQNSALA

SEQ ID NO: 60 - DNA, 57 bp, Pichia pastoris
atgagcaccctgacattgctggctgtgctgttgtcgcttcaaaattcagctcttgct

SEQ ID NO: 61 - protein, 22 aa, Pichia pastoris
MQFNWNIKTVASILSALTLAQA

SEQ ID NO: 62 - DNA, 66 bp, Pichia pastoris
atgcaattcaactggaatattaaaactgtggcaagtattttgtccgctctcacactagcacaagca

SEQ ID NO: 63 - protein, 18 aa, Pichia pastoris
MKLLSLVSIAATTALAKA

SEQ ID NO: 64 - DNA, 54 bp, Pichia pastoris
atgaaattgttatcattagtatctattgctgctacaactgcgctagcaaaagct

SEQ ID NO: 65 - DNA, 600 bp, Pichia pastoris
tcacattctttcactctacaaaatgaccagagtacgaaatatacgcatacattcgattcaagttttttaaagccttacatcgtatgtctggcaaaatcagagaatgcctcgtgaaagaaaaagactgaatccattaacttgcatgccaactcaatcccgactgtcaatcattcatccttgcgtcttttgaacatctatgcttccacaagtcaattcttgatttagtatacacataaccaaatttggatcaagtttgaagtaaaactttaacttcagctccttacatttgcactaagatctctgctactctggtcccaagtgaaccaccttttggaccctattgaccggaccttaacttgccaaacctaaacgcttaatgcctcagacgttttaatgcctctcaacacctccaaggttgctttcttgagcatgcctactaggaactttaacgaactgtggggttgcagacagtttcaggcgtgtcccgaccaatatggcctactagactctctgaaaaatcacagttttccagtagttccgatcaaattaccatcgaaatggtcccataaacggacatttgacatccgttcctgaattata

SEQ ID NO: 66 - protein, 16 aa, Artificial sequence
  Synthetic polypeptide
MQYIKANSKFIGITEL

(19) United States
(12) Patent Application Publication
(10) Pub. No.: US 2020/0032279 A1
(43) Pub. Date: Jan. 30, 2020

(54) CONSTRUCTS AND CELLS FOR ENHANCED PROTEIN EXPRESSION

(71) Applicant: Massachusetts Institute of Technology, Cambridge, MA (US)

(72) Inventors: Kerry R. Love, Somerville, MA (US); J. Christopher Love, Somerville, MA (US); Charles Whittaker, Winthrop, MA (US); Joseph Brady, Cambridge, MA (US); Catherine Bartlett Matthews, Cambridge, MA (US); Noelle Colant, Akron, OH (US); Neil C. Dalvie, Cambridge, MA (US)

(73) Assignee: Massachusetts Institute of Technology, Cambridge, MA (US)

(21) Appl. No.: 16/080,844

(22) PCT Filed: Jan. 10, 2018
(86) PCT No.: PCT/US2018/013220

§ 371 Date: Aug. 29, 2018

Related U.S. Application Data
(60) Provisional application No. 62/444,758, filed on Jan. 10, 2017.

(51) Int. Cl. C12N 15/81; C12P 21/02

(52) U.S. Cl. CPC C12N 15/81; C12P 21/02

(57) ABSTRACT

Described are expression constructs, cells, and methods of producing proteins in Pichia pastoris.

### RELATED APPLICATION

This application claims the benefit of the filing date of U.S. Provisional Application No. 62/444,758, filed on Jan. 10, 2017, the content of which is herein incorporated by reference in its entirety.

### BACKGROUND OF THE INVENTION

Biopharmaceuticals, including recombinant therapeutic proteins, nucleic acid products, and therapies based on engineered cells, represent an important public health need. Despite major advances, the price, affordability, and ease of production remain obstacles to ubiquitous access to groundbreaking therapies. In biomanufacturing, a significant cost driver is product titer, or produced concentration of functional product. All current industrial cell hosts contain weaknesses in which improvement would enhance the production of biologics.

Current industrial cell hosts include E. coli, Chinese Hamster Ovary (CHO) cells, and S. cerevisiae, which combine to produce nearly all marketed biologics. E. coli offers a fast and inexpensive host but production of proteins of eukaryotic hosts can be problematic. CHO cells are capable of human-like post-translational modifications but are slow to grow, inconsistent in reproducibility, require expensive media for growth, and produce proteins that can be difficult to purify. S. cerevisiae also possesses eukaryotic post-translational machinery; however, excess mannose sugar residues are added, sometimes resulting in immunogenicity and toxicity and recovery of these proteins often requires whole-cell lysis, complicating purification. Thus, a need exists to engineer new types of host cells to produce proteins efficiently.

### SUMMARY OF THE INVENTION

The invention provides expression constructs, cells expressing heterologous proteins, and methods of producing heterologous proteins. In one aspect, the invention features an expression construct including an OLE1 promoter operably linked to a nucleic acid encoding a polypeptide including a signal sequence and a heterologous protein. In a related aspect, the invention features a methylotrophic cell expressing a heterologous protein, wherein the expression is under the control of an OLE1 promoter. In some embodiments, the OLE1 promoter is located at an OLE1, AOX1, GAPDH, DAS2, or PIF1 locus. The methylotrophic cell may be transformed using an expression construct of the invention. In some embodiments, the OLE promoter has at least 95% (e.g. 95%, 96%, 97%, 98%, 99%, or 100%) homology with SEQ ID NO: 1 or a protein-expressing fragment thereof.

In another aspect, the invention features an expression construct including a DAS2 promoter operably linked to a nucleic acid encoding a polypeptide including a signal sequence and a heterologous protein and a targeting sequence for integration in a methylotrophic cell at a non-native locus. In a related aspect, the invention features a methylotrophic cell expressing a heterologous protein, wherein the expression is under the control of a DAS2 promoter integrated at a non-native locus, e.g., an OLE1, AOX1, GAPDH, or PIF1 locus. The methylotrophic cell may be transformed using an expression construct of the invention. In some embodiments, the DAS2 promoter has at least 95% (e.g. 95%, 96%, 97%, 98%, 99%, or 100%) homology with SEQ ID NO: 2 or a protein-expressing fragment thereof.

In another aspect, the invention features an expression construct including an AOX1 promoter operably linked to a nucleic acid encoding a polypeptide including a signal sequence and a heterologous protein, the construct further including a targeting sequence for integration in a methylotrophic cell at a PIF1, OLE1, or DAS2 locus. In a related aspect, the invention features a methylotrophic cell expressing a heterologous protein, wherein the expression is under the control of an AOX1 promoter integrated at a PIF1, OLE1, or DAS2 locus. The methylotrophic cell may be transformed using an expression construct of the invention. In some embodiments, the AOX1 promoter has at least 95% (e.g. 95%, 96%, 97%, 98%, 99%, or 100%) homology with SEQ ID NO: 3 or a protein-expressing fragment thereof.

In another aspect, the invention features an expression construct including a GAPDH promoter operably linked to a nucleic acid encoding a polypeptide including a signal sequence and a heterologous protein, the construct further including a targeting sequence for integration in a cell at an AOX1, PIF1, OLE1, or DAS2 locus. In a related aspect, the invention features a cell, e.g., a yeast cell or methylotrophic cell, expressing a heterologous protein, wherein the expression is under the control of a GAPDH promoter integrated at an AOX1, PIF1, OLE1, or DAS2 locus. The cell may be transformed using an expression construct of the invention. In some embodiments, the GAPDH promoter has at least 95% (e.g. 95%, 96%, 97%, 98%, 99%, or 100%) homology with SEQ ID NO: 4 or a protein-expressing fragment thereof.

In some embodiments of any of the above aspects, the signal sequence is identical to the signal sequence of a naturally occurring yeast protein such as SCW11, MSC1, EXG1, 0841, 1286, BGL2, 2488, 2848, PRY2, 4355, PIR1 KAR2, TOS1, 2241, LHS1, TIF1, CTS1, or 5326, e.g., KAR2, MSC1, TOS1, 2241, LHS1, TIF1, CTS1, or 5326.

In another aspect, the invention features an expression construct including a promoter operably linked to a nucleic acid encoding a polypeptide including a signal sequence and a heterologous protein, wherein the signal sequence is a signal sequence of KAR2, MSC1, TOS1, 2241, LHS1, TIF1, CTS1, or 5326. In some embodiments, the promoter is an OLE1, AOX1, DAS2, or GAPDH promoter. In some embodiments, the expression construct includes a targeting sequence for integration in a methylotrophic cell at an AOX1, PIF1, OLE1, GAPDH, or DAS2 locus. In a related aspect, the invention features a methylotrophic cell expressing a heterologous protein fused to a signal sequence of KAR2, MSC1, TOS1, 2241, LHS1, TIF1, CTS1, or 5326. In some embodiments, the expression is under the control of an OLE1, AOX1, DAS2, or GAPDH promoter. In some embodiments, the heterologous protein is integrated at an AOX1, PIF1, OLE1, GAPDH, or DAS2 locus.

In another aspect, the invention features an expression construct comprising a promoter operably linked to a nucleic acid encoding a polypeptide comprising a signal sequence and a heterologous protein, wherein (i) the promoter is an AOX1 or DAS2 promoter and/or the construct further comprises a targeting sequence for integration in a methylotrophic cell at an AOX1 or DAS2 locus; (ii) the expression construct further comprises a Kozak sequence beginning at the −3 position relative to the translation start site of the nucleic acid encoding the polypeptide; and/or (iii) a mRNA secondary structure of the nucleic acid encoding a polypeptide has been reduced or eliminated relative to the endogenous mRNA encoding the heterologous protein. In a related aspect, the invention features a cell, e.g., a yeast cell or methylotrophic cell, expressing a heterologous protein under the control of a promoter, wherein (i) the promoter is an AOX1 promoter or a DAS2 promoter and/or the promoter is located at an AOX1 or DAS2 locus; (ii) mRNA encoding the heterologous protein comprises a Kozak sequence beginning at the −3 position relative to the translation start site; and/or (iii) a mRNA secondary structure of the mRNA encoding the heterologous protein has been reduced or eliminated relative to the endogenous mRNA encoding the heterologous protein.

In another aspect, the invention features a method for preparing a transgene expression construct for expressing a heterologous protein in Pichia comprising providing a nucleic acid encoding a heterologous protein; and (i) selecting a promoter that increases expression of genes of the Mut pathway upon integration; or (ii) selecting a targeting sequence for guided recombination into a locus, wherein insertion of the heterologous protein into the locus increases expression of genes of the Mut pathway; or (i) and (ii).

In some embodiments of any of the above aspects, an expression construct of the invention is a plasmid or viral vector. The plasmid may be an episomal plasmid or an integrative plasmid. The expression construct may be linearized (e.g. by a restriction enzyme).

In another aspect, the invention features a method of producing a heterologous protein with a methylotrophic cell. The method includes culturing the cell under conditions suitable to express the heterologous protein. In some embodiments, the method includes first culturing the cell with a first carbon source lacking methanol under conditions in which the heterologous protein is substantially not expressed, followed by switching the carbon source to a carbon source that includes methanol to express the heterologous protein. In some embodiments, the method further includes isolating the protein. In other embodiments, the method further includes transforming the methylotrophic cell with an expression construct encoding the heterologous protein, as described herein.

In embodiments of any of the above aspects, the heterologous protein is selected from the group consisting of enzymes, hormones, antibodies or antigen binding fragments thereof, vaccine components, blood factors, thrombolytic agents, cytokines, receptors, and fusion proteins. In further embodiments of any of the above aspects, the methylotrophic cell is a yeast cell, such as a Pichia pastoris, Komagataella phaffii or Komagataella pastoris cell. The Komagataella phaffii cell may be a Komagataella phaffii Y-11430, Y-7556, YB-4290, Y-12729, Y-17741, Y-48123, Y-48124, YB-378, YB-4289, GS115, KM71H, SMD1168, SMD1168H, or X-33 cell.

In some embodiments of any of the above aspects, the expression construct comprises a Kozak sequence beginning at the −3 position relative to the translation start site of the nucleic acid encoding the polypeptide. In some embodiments, the mRNA encoding the heterologous protein comprises a Kozak sequence beginning at the −3 position relative to the translation start site. In some embodiments, the Kozak sequence comprises (i) the sequence ANAATGNC, wherein N comprises A, T, G, or C; or (ii) the sequence AMMATG, wherein M comprises A or C.

In some embodiments of any of the above aspects, a mRNA secondary structure of the nucleic acid encoding a polypeptide or of the has been reduced or eliminated relative to the endogenous mRNA encoding the polypeptide. In some embodiments, a mRNA secondary structure of the mRNA encoding the heterologous protein has been reduced or eliminated relative to the endogenous mRNA encoding the heterologous protein. In some embodiments, the mRNA secondary structure is selected from a hairpin loop or any other structure as predicted by likelihood of pairing and/or low free energy.

### BRIEF DESCRIPTION OF THE DRAWINGS

FIG. 1 is a schematic diagram showing a plasmid used for integration at the AOX1 promoter. In the right panel, is a schematic diagram showing how the linearized plasmid is integrated into the host genome via homologous recombination.

FIG. 2 is a set of graphs showing RNA expression of genes as a function of glycerol or glucose versus methanol as the primary carbon source.

FIG. 3 is a heat map that quantifies the expression of representative genes under glycerol or methanol conditions.

FIG. 4 is a bar graph that shows the titer of human growth hormone (hGH) expression when the hGH gene is expressed under various promoters at various loci.

FIG. 5 is an image of an immunoblot experiment showing hGH expression under various promoters at their native or AOX1 loci.

FIG. 6 is a graph quantifying the ratio of secreted protein in glycerol versus methanol normalized by total gene expression in glycerol as measured by RNA-seq.

FIG. 7 is an image of a dot blot experiment showing the expression of a protein with eleven different signal sequences.

FIG. 8A-8B includes data showing the effect of the DAS2 promoter and the AOX1 promoter at various loci on gene expression. FIG. 8A is a graph showing hGH titer at 24 hr post-induction as a function of cassette copy number for P_{DAS2} and P_{AOX1} strains. FIG. 8B is a heatmap comparing expression of methanol utilization pathway (Mut) genes across high-producing strains. DAS2 strains display upregulated Mut, particularly of DAS1 and DAS2 strains, relative to other high-producers.

FIG. 9A-9B shows a comparison of 5′ untranslated region (UTR) sequences and translation efficiencies for hGH versus the consensus Kozak sequence in P. pastoris. FIG. 9A is a HMM Logo of the Kozak sequence across all P. pastoris genes depicting preference for A(A/C)(A/C)ATG. FIG. 9B is a chart showing the −4 to +3 sequence and translation efficiency for each promoter/5′UTR used to direct heterologous hGH gene expression. The highlighted 5′UTR's indicate −3 nucleotide match to consensus.

FIG. 10 includes data showing the effect of codon optimization that mitigates mRNA hairpin formation on expression of full length VP8* and on expression of N-terminally truncated VP8* variants. The top diagram depicts the desired full length VP8* protein consists of residues 86 through 265, directly following the alpha mating factor (uMF) signal sequence. The diagram in the bottom left shows predicted mRNA secondary structures that alter the N-terminus of secreted heterologous proteins (VP8* variants depicted). V1, V2, V3 and V4 represent N-terminal VP8* variants (N-terminally truncated proteins), which correlate with the existence of the hairpin shown on the bottom left. For the bar graph on the bottom right, Alt1 has codons 6, 8, 15, and 16 altered (4 changes), Alt2 has codons 6, 8, 9, 15, and 16 altered (5 changes), Alt3 has codons 6, 8, 9, 15, 16, 21 altered (6 changes).

### DETAILED DESCRIPTION

The invention provides expression constructs and methylotrophic cells that express heterologous proteins, as well as methods to produce heterologous proteins. The cells advantageously produce a significantly higher titer of heterologous protein compared to prior expression systems. The DNA constructs are designed to drive gene expression under the control of highly active methanol-inducible promoters and can be integrated at various loci in the genome that enhance protein production. Furthermore, signal sequences of efficiently secreted proteins can be incorporated into the constructs to produce cells resulting in an increase in the titer of protein produced.

### Definitions

By “expression construct” is meant a nucleic acid construct including a promoter operably linked to a nucleic acid sequence of a heterologous protein. Other elements may be included as described herein and known in the art.

By “integration” is meant insertion of a nucleotide sequence into a host cell chromosome or episomal DNA element, such as by homologous recombination.

By “methylotrophic cell” is meant a cell having the ability to use reduced one-carbon compounds, such as methanol or methane, as a carbon source for cellular growth.

By “operably linked” is meant that a gene and a regulatory sequence(s) (e.g., a promoter) are connected in such a way as to permit gene expression when the appropriate molecules (e.g., transcriptional activator proteins) are bound to the regulatory sequence(s).

By “protein” is meant any chain of amino acids, regardless of length or post-translational modification (e.g., glycosylation or phosphorylation). For the purposes of this invention, a “heterologous protein” is a protein not natively expressed by a methylotrophic cell, e.g., a mammalian protein, such as a human protein.

By “promoter” is meant a DNA sequence sufficient to direct transcription; such elements may be located in the 5′ region of the gene. An OLE1 promoter is one having at least 80% homology to SEQ ID NO.: 1 or any protein-expressing fragment thereof and producing at least 80% of the heterologous protein as SEQ ID NO: 1 under the same conditions. A DAS2 promoter is one having at least 80% homology to SEQ ID NO.: 2 or any protein-expressing fragment thereof and producing at least 80% of the heterologous protein as SEQ ID NO: 2 under the same conditions. An AOX1 promoter is one having at least 80% homology to SEQ ID NO.: 3 or any protein-expressing fragment thereof and producing at least 80% of the heterologous protein as SEQ ID NO: 3 under the same conditions. A GAPDH promoter is one having at least 80% homology to SEQ ID NO.: 4 or any protein-expressing fragment thereof and producing at least 80% of the heterologous protein as SEQ ID NO: 4 under the same conditions.

By “signal sequence” is meant a short peptide present at the N-terminus of a newly synthesized heterologous protein that directs the protein toward the secretory pathway of a cell. The signal sequence is typically cleaved from the heterologous protein prior to secretion.

The term “nucleic acid,” in its broadest sense, includes any compound and/or substance that comprises a polymer of nucleotides. These polymers are referred to as polynucleotides.

Nucleic acids (also referred to as polynucleotides) may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. In some preferred embodiments, an mRNA is translated in vivo.

The basic components of an mRNA molecule typically include at least one coding region, a 5′ untranslated region (UTR), a 3′ UTR, a 5′ cap and a poly-A tail.

### Methylotrophic Cells

An exemplary methylotrophic cell for use in the present invention is a yeast cell, such as Pichia pastoris, which offers an attractive blend of advantages as a host for protein production. Two useful P. pastoris strains include Komagataella pastoris and Komagataella phaffii. As a eukaryotic organism, it is capable of producing the complex post-translational modifications required for human biologics, and it exhibits fast, robust growth on inexpensive media. It possesses a small, tractable 9.4 MB genome that can be easily manipulated with an established toolbox of genetic techniques. Examples of strains of K. phaffii include NRRL Y-11430, Y-7556, YB-4290, Y-12729, Y-17741, Y-48123, Y-48124, YB-378, YB-4289, GS115, KM71H, SMD1168, SMD1168H, and X-33.

Heterologous proteins can be expressed in methylotrophic cells using a promoter at either native locus or an alternate locus and a source of carbon, e.g., methanol. In the context of the present invention, such promoters include OLE1, DAS2, AOX1, and GAPDH promoters.

### Expression Constructs

Expression constructs can provide an early and inexpensive opportunity for optimization of protein quality and titer. High-quality protein is properly folded and full-length (intact), with native N- and C-termini, and without significant proteolysis. In engineering the expression constructs, factors such as the promoter for heterologous gene expression, target site for transgene integration, sequence for translation initiation, and mRNA codon-optimization of the gene of interest are important design points for a given protein-expressing strain.

Expression constructs are nucleic acid constructs that minimally include a promoter or any protein-expressing fragment thereof operably linked to a nucleotide sequence for a heterologous protein. Expression constructs may also include additional elements as is described herein and known in the art. In some embodiments, the expression construct can include one or more of any of the following components: signal sequence, targeting sequence, transcription terminator sequence, origin of replication, multi-cloning site, and an antibiotic resistance marker (which is optionally under the control of its own promoter, e.g., TEFI or GAPDH). In some embodiments, the construct is a viral vector or a plasmid, such as an episomal plasmid or an integrative plasmid. In some embodiments, the construct comprises a transgene cassette. Transgene cassettes may include, e.g., a promoter, a nucleotide sequence for a heterologous protein of interest, and a terminator. Transgene cassettes may also include, e.g., a targeting sequence for guided recombination and/or a selective marker for isolation of positive clones. The construct can be linearized e.g., with a restriction enzyme or it can be in closed-circular form. The construct can be used to transform a methylotrophic cell (e.g. yeast) by electroporation, heat shock, or chemical transformation with lithium acetate. Once integrated, the altered genome is preferably passed on to each replicative generation.

Efforts to-date regarding selection of loci for transgene cassette insertion have focused primarily on locus accessibility for expressing the gene of interest. However, this disclosure demonstrates that use of certain promoters may upregulate native (endogenous) genes (e.g., coding regions) and provide an unexpected benefit to cell health and metabolism that results in increased titers and/or quality of heterologous proteins. This includes, but is not limited to, upregulation of the DAS1, DAS2, AOX1, GAPDH, and ATG30 genes by use of the respective promoter or locus. In the case of DAS1, DAS2, and AOX1, upregulating these genes can upregulate the overall Mut pathway. Since the organism relies on methanol as its carbon source during the production phase of fermentation, enhanced utilization by upregulation of the Mut pathway enables greater cell productivity. It was unexpected that use of a Mut pathway promoter or locus can drive significant upregulation of this pathway.

In some embodiments, expression of the heterologous protein from the promoter and/or at the loci results in an increase or decrease in expression of one or more endogenous genes. In some embodiments, expression of the heterologous protein from the promoter and/or at the loci results in an upregulation of expression of one or more genes in the Mut pathway. In some embodiments, one or more genes in the Mut pathway are upregulated at least 2-fold, at least 3-fold, at least 4-fold, at least 5-fold, at least 10-fold, at least 50-fold, at least 100-fold, at least 1000-fold compared to cells that do not have the heterologous protein inserted.

Exemplary promoters include OLE1, DAS2, AOX1, and GAPDH promoters. These promoter sequences may have at least 80% homology to SEQ ID NOs.: 1-4 (e.g., identical to SEQ ID NOs: 1-4) or any protein-expressing fragment thereof. For example, the promoter sequence may have at least 85, 90, 95, or 99% homology to one of SEQ ID NOs.: 1-4 or any protein-expressing fragment thereof. For a promoter not identical to one of SEQ ID NOs.: 1-4 or any protein-expressing fragment thereof, the promoter will result in protein expression of at least 80% of the protein expressed under control of the corresponding wild type sequence under the same conditions. For example, a promoter sequence or any protein-expressing fragment thereof with less than 100% homology to one of SEQ ID Nos.: 1-4 may result in protein expression of at least 85, 90 95, or 99% of the protein expressed under control of the corresponding wild type sequence under the same conditions.

OLE1 promoter
SEQ ID NO: 1
GATAAAAAAAAACGAGACGATAAGATGAGGAAGGTACCACACATGGGCATTCTTAG
TGCGCGAGAGATGATTAGCATCGAGGGAAAGCTTAAACATCTTTGGTCTACGTAAG
CAGAGACCAGGCACTAGCAAGCCTAATTAGGGTTAGGGAATTGAATGTCAGCAAAA
GCTGAGGCGGCTTCCGAGGGCCAATAGAATAAGAAAGAACAACTTAGGGCGCAAAC
CTGATTGCGATTTTGGGGCTTTCCTTGGAAAAGACTTGATCCCTACGCTGTGGAAGG
CGCACTACTATCGAAGCTCCCTCTAACCTCCCAAAGGAGAAGGAAGGGAAAAAAAA
ATAGTGACAAAAAGAAAACAAAGAGCCCAAGACCTCTATCGCCCCATCGCCCAGAT
CTCCTATCAGCAAAATTATGTAAGCTGCATCTTTTGGTGAGCTAAAGGGGACTTTCG
CGCTAACAAAAAGAGCAAACTTGTTTGTTGGGTGATTGTTGGGTGTTCAAGGCACGA
CTTTCTAATCTACCTTGCATTGACAGATTCTTCCAACTGCGCCCGATATAACGTAGCA
TTGCCAGGTAATGATGGTATACTTTACATGGTCACACTACGACGCTCAACATCAGTC
CCTCTTAGTGGAACCACAACTTGCTCGTTGAATTTTGGAGCGTAATGTGTCATGTTG
GGTCCTGCAAAAAGAAAAGTTGGATCCCATAAATTTAGACTTTGTAGGATGACAATC
TACAGAGATTTCTCGAACTTCGGGCCTTCCTATAAAACAAGATAAACTCCTTCCTCTT
TCTCTTTCCTTCTCTTTAGTCTTCTCACTTCATCTACGCCACACA
DAS2 promoter
SEQ ID NO: 2
ATTACTGTTTTGGGCAATCCTGTTGATAAGACGCATTCTAGAGTTGTTTCATGAAAG
GGTTACGGGTGTTGATTGGTTTGAGATATGCCAGAGGACAGATCAATCTGTGGTTTG
CTAAACTGGAAGTCTGGTAAGGACTCTAGCAAGTCCGTTACTCAAAAAGTCATACCA
AGTAAGATTACGTAACACCTGGGCATGACTTTCTAAGTTAGCAAGTCACCAAGAGG
GTCCTATTTAACGTTTGGCGGTATCTGAAACACAAGACTTGCCTATCCCATAGTACA
TCATATTACCTGTCAAGCTATGCTACCCCACAGAAATACCCCAAAAGTTGAAGTGAA
AAAATGAAAATTACTGGTAACTTCACCCCATAACAAACTTAATAATTTCTGTAGCCA
ATGAAAGTAAACCCCATTCAATGTTCCGAGATTTAGTATACTTGCCCCTATAAGAAA
CGAAGGATTTCAGCTTCCTTACCCCATGAACAGAAATCTTCCATTTACCCCCCACTG
GAGAGATCCGCCCAAACGAACAGATAATAGAAAAAAGAAATTCGGACAAATAGAA
CACTTTCTCAGCCAATTAAAGTCATTCCATGCACTCCCTTTAGCTGCCGTTCCATCCC
TTTGTTGAGCAACACCATCGTTAGCCAGTACGAAAGAGGAAACTTAACCGATACCTT
GGAGAAATCTAAGGCGCGAATGAGTTTAGCCTAGATATCCTTAGTGAAGGGTTGTTC
CGATACTTCTCCACATTCAGTCATAGATGGGCAGCTTTGTTATCATGAAGAGACGGA
AACGGGCATTAAGGGTTAACCGCCAAATTATATAAAGACAACATGTCCCCAGTTTA
AAGTTTTTCTTTCCTATTCTTGTATCCTGAGTGACCGTTGTGTTTAATATAACAAGTT
CGTTTTAACTTAAGACCAAAACCAGTTACAACAAATTATAACCCCTCTAAACACTAA
AGTTCACTCTTATCAAACTATCAAACATCAAAA
AOX1 promoter
SEQ ID NO: 3
AGATCTAACATCCAAAGACGAAAGGTTGAATGAAACCTTTTTGCCATCCGACATCCA
CAGGTCCATTCTCACACATAAGTGCCAAACGCAACAGGAGGGGATACACTAGCAGC
AGACCGTTGCAAACGCAGGACCTCCACTCCTCTTCTCCTCAACACCCACTTTTGCCA
TCGAAAAACCAGCCCAGTTATTGGGCTTGATTGGAGCTCGCTCATTCCAATTCCTTCT
ATTAGGCTACTAACACCATGACTTTATTAGCCTGTCTATCCTGGCCCCCCTGGCGAG
GTTCATGTTTGTTTATTTCCGAATGCAACAAGCTCCGCATTACACCCGAACATCACTC
CAGATGAGGGCTTTCTGAGTGTGGGGTCAAATAGTTTCATGTTCCCCAAATGGCCCA
AAACTGACAGTTTAAACGCTGTCTTGGAACCTAATATGACAAAAGCGTGATCTCATC
CAAGATGAACTAAGTTTGGTTCGTTGAAATGCTAACGGCCAGTTGGTCAAAAAGAA
ACTTCCAAAAGTCGGCATACCGTTTGTCTTGTTTGGTATTGATTGACGAATGCTCAA
AAATAATCTCATTAATGCTTAGCGCAGTCTCTCTATCGCTTCTGAACCCCGGTGCACC
TGTGCCGAAACGCAAATGGGGAAACACCCGCTTTTTGGATGATTATGCATTGTCTCC
ACATTGTATGCTTCCAAGATTCTGGTGGGAATACTGCTGATAGCCTAACGTTCATGA
TCAAAATTTAACTGTTCTAACCCCTACTTGACAGCAATATATAAACAGAAGGAAGCT
GCCCTGTCTTAAACCTTTTTTTTTATCATCATTATTAGCTTACTTTCATAATTGCGACT
GGTTCCAATTGACAAGCTTTTGATTTTAACGACTTTTAACGACAACTTGAGAAGATC
AAAAAACAACTAATTATTCGAAACG
GAPDH promoter
SEQ ID NO: 4
AGATCTTTTTTGTAGAAATGTCTTGGTGTCCTCGTCCAATCAGGTAGCCATCTCTGAA
ATATCTGGCTCCGTTGCAACTCCGAACGACCTGCTGGCAACGTAAAATTCTCCGGGG
TAAAACTTAAATGTGGAGTAATGGAACCAGAAACGTCTCTTCCCTTCTCTCTCCTTCC
ACCGCCCGTTACCGTCCCTAGGAAATTTTACTCTGCTGGAGAGCTTCTTCTACGGCC
CCCTTGCAGCAATGCTCTTCCCAGCATTACGTTGCGGGTAAAACGGAGGTCGTGTAC
CCGACCTAGCAGCCCAGGGATGGAAAAGTCCCGGCCGTCGCTGGCAATAATAGCGG
GCGGACGCATGTCATGAGATTATTGGAAACCACCAGAATCGAATATAAAAGGCGAA
CACCTTTCCCAATTTTGGTTTCTCCTGACCCAAAGACTTTAAATTTAATTTATTTGTCC
CTATTTCAATCAATTGAACAACTAT

The heterologous protein expressed by a methylotrophic cell of the invention can be any non-natively expressed protein. Such proteins may be native to another species or artificial and include enzymes (such as trypsin or imiglucerase), hormones (e.g., insulin, glucagon, human growth hormone, gonadotrophins, erythropoietin, or a colony stimulating factor), antibodies or antigen binding fragments thereof (e.g., a monoclonal antibody or Fab fragment), single chain variable fragments (scFvs), nanobodies, a vaccine component, a blood factor (e.g., Factor VIII or Factor IX), a thrombolytic agent (e.g., tissue plasminogen activator), cytokines (such as interferons (e.g., interferon-α, -β, or -γ), interleukins (e.g., IL-2) and tumor necrosis factors), receptors, and fusion proteins (e.g., receptor fusions).

Typically, the heterologous protein will be expressed with a signal sequence. The signal sequences may be expressed under the control of any of the promoters described herein or other suitable promoters, e.g., any methanol inducible promoter. A signal sequence is a short peptide present at the N-terminus of newly synthesized proteins. The peptide directs the proteins toward the secretory pathway and is typically cleaved from the heterologous protein prior to secretion. Examples of signal sequences that may be employed in this invention are shown in Table 1. It will be understood that other nucleic acid sequences may be employed that result in the same protein sequence because of the degeneracy of the genetic code. Signal sequences producing a peptide with at least 80% homology to those listed in Table 1 may be employed. For example, signal sequences may produce a peptide having at least 85, 90, 95, or 99% homology to a peptide listed in Table 1. In certain embodiments, the signal sequence is one of KAR2, MSC1, TOS1, 2241, LHS1, TIF1, CTS1, and 5326. Other signal sequences are known in the art, e.g., alpha mating factor (MFα) from S. cerevisiae.

TABLE 1
Exemplary signal sequences

| | Gene | | | SEQ ID NO. |
|---|---|---|---|---|
| Gene ID | Name | Signal Peptide | Nucleic Acid Sequence | (protein/DNA) |
| GQ67_00077 | SCW11 | MLSTILNIFILLLFI | ATGCTATCAACTATCTTAAATATCTTTATCCTGTTG | 5/6 |
| | | QASLQ | CTCTTCATACAGGCATCCCTACAG | |
| GQ67_00168 | KAR2 | MLSLKPSWLTLAA | ATGCTGTCGTTAAAACCATCTTGGCTGACTTTGGCG | 7/8 |
| | | LMYAMLLVVVPF | GCATTAATGTATGCCATGCTATTGGTCGTAGTGCC | |
| | | AKPVRA | ATTTGCTAAACCTGTTAGAGCT | |
| GQ67_00198 | 0198 | MFLKSLLSFASILT | ATGTTCCTCAAAAGTCTCCTTAGTTTTGCGTCTATC | 9/10 |
| | | LCKA | CTAACGCTTTGCAAGGCC | |
| GQ67_00220 | MSC1 | MRIFHWILFFITTS | ATGAGAATTTTTCACTGGATTCTCTTCTTTATTACC | 11/12 |
| | | LA | ACTTCGCTTGCC | |
| GQ67_00497 | EXG1 | MNLYLITLLFASLC | ATGAACTTGTACCTAATTACATTACTATTCGCCAGT | 13/14 |
| | | SA | CTATGCAGCGCA | |
| GQ67_00591 | 0591 | MSYLKISALLSVLS | ATGTCTTACTTGAAAATTTCCGCTTTGCTTTCAGTT | 15/16 |
| | | VALA | TTGTCCGTCGCCTTGGCC | |
| GQ67_00841 | 0841 | MMYRNLIIATALT | ATGATGTACAGGAACTTAATAATTGCTACTGCCCT | 17/18 |
| | | CGAYS | TACTTGCGGTGCATACAGT | |
| GQ67_01286 | 1286 | MKISALTACAVTL | ATGAAGATATCCGCTCTTACAGCCTGCGCTGTTACT | 19/20 |
| | | AGLAIA | CTAGCTGGTCTTGCAATTGCA | |
| GQ67_01384 | TOS1 | MKLSATLLLSVFT | ATGAAGTTATCAGCAACCTTACTGCTCTCCGTTTTC | 21/22 |
| | | SIQSAYA | ACTTCCATCCAGTCTGCCTACGCT | |
| GQ67_01735 | BGL2 | MIFNLKTLAAVAIS | ATGATCTTTAATCTTAAAACACTGGCTGCGGTTGC | 23/24 |
| | | ISQVSA | AATCTCCATTTCACAAGTGTCTGCA | |
| GQ67_02241 | 2241 | MSCLSHLIASVCFL | ATGAGTTGTTTATCCCATCTTATCGCTAGCGTATGT | 25/26 |
| | | LCIVEA | TTTTTGTTATGCATAGTAGAAGCT | |
| GQ67_02314 | LHS1 | MRTQKIVTVLCLL | ATGAGAACACAAAAGATAGTAACAGTACTTTGTTT | 27/28 |
| | | LNTVLG | GCTACTAAATACTGTGCTTGGA | |
| GQ67_02485 | GAS1 | MLIGSCLLSSVLA | ATGTTAATAGGATCCTGCCTATTGAGTTCAGTCTTG | 29/30 |
| | | | GCA | |
| GQ67_02486 | 2486 | MLSILSALTLLGLS | ATGTTGTCCATTTTAAGTGCATTAACTCTGCTGGGC | 31/32 |
| | | CA | CTGTCTTGTGCT | |
| GQ67_02488 | 2488 | MQVKSIVNLLLAC | ATGCAAGTTAAATCTATCGTTAACCTACTGTTGGC | 33/34 |
| | | SLAVA | ATGTTCGTTGGCCGTGGCC | |
| GQ67_02707 | DSE4 | MSFSSNVPQLFLLL | ATGTCATTCTCTTCCAACGTGCCACAACTTTTCTTG | 35/36 |
| | | VLLTNIVSG | TTGTTGGTTCTGTTGACCAATATAGTCAGTGGA | |
| GQ67_02848 | 2848 | MKLLNFLLSFVTL | ATGAAATTGTTGAACTTTCTGCTTAGCTTCGTAACT | 37/38 |
| | | FGLLSGSVFA | CTGTTCGGACTATTATCAGGTTCTGTGTTTGCA | |
| GQ67_03026 | FLO9- | MKFPVPLLFLLQL | ATGAAATTTCCTGTGCCACTTTTGTTTCTACTGCAG | 39/40 |
| | like2 | FFIIATQG | CTGTTCTTTATTATTGCAACACAAGGA | |
| GQ67_03041 | 3041 | MKFAISTLLIILQA | ATGAAGTTCGCAATTTCAACACTTCTTATTATCCTA | 41/42 |
| | | AAVFA | CAGGCTGCCGCTGTTTTTGCT | |
| GQ67_03092 | PRY2 | MKLSTNLILAIAA | ATGAAGCTCTCCACCAATTTGATTCTAGCTATTGCA | 43/44 |
| | | ASAVVSA | GCAGCTTCCGCCGTTGTCTCAGCT | |
| GQ67_03672 | TIF1 | MHPYTVVFARLLL | ATGCATCCATACACCGTAGTATTTGCGCGCCTCCTC | 45/46 |
| | | GVFSTA | CTGGGTGTTTTCTCAACTGCC | |
| GQ67_04133 | CTS1 | MKFFYFAGFISLLQ | ATGAAATTTTTTTACTTTGCGGGGTTCATATCTCTG | 47/48 |
| | | LIFA | TTACAGCTGATATTCGCC | |
| GQ67_04226 | PEP4 | MIFDGTTMSIAIGL | ATGATATTTGACGGTACTACGATGTCAATTGCCATT | 49/50 |
| | | LSTLGIGAEA | GGTTTGCTCTCTACTCTAGGTATTGGTGCTGAAGCC | |
| GQ67_04355 | 4355 | MKSQLIFMALASL | ATGAAATCTCAACTTATCTTTATGGCTCTTGCCTCT | 51/52 |
| | | VAS | CTGGTGGCCTCC | |
| GQ67_04638 | PIR1 | MKLAALSTIALTIL | ATGAAGCTCGCTGCACTCTCCACTATTGCATTAACT | 53/54 |
| | | PVALA | ATTTTACCCGTTGCCTTGGCT | |
| GQ67_04640 | YMR24 | MQFNSVVISQLLL | ATGCAATTCAACAGTGTCGTCATCAGCCAACTTTT | 55/56 |
| | 4W | TLASVSMG | GCTGACTCTAGCCAGTGTCTCAATGGGA | |
| GQ67_04929 | CRH1 | MVSLTRLLVTGIA | ATGGTTTCTTTAACAAGACTACTAGTTACCGGAAT | 57/58 |
| | | TALQVNA | CGCCACCGCTTTGCAGGTGAATGCC | |
| GQ67_05018 | 5018 | MSTLTLLAVLLSL | ATGAGCACCCTGACATTGCTGGCTGTGCTGTTGTC | 59/60 |
| | | QNSAL A | GCTTCAAAATTCAGCTCTTGCT | |
| GQ67_05237 | PDI1 | MQFNWNIKTVASI | ATGCAATTCAACTGGAATATTAAAACTGTGGCAAG | 61/62 |
| | | LSALTLAQA | TATTTTGTCCGCTCTCACACTAGCACAAGCA | |
| GQ67_05326 | 5326 | MKLLSLVSIAATT | ATGAAATTGTTATCATTAGTATCTATTGCTGCTACA | 63/64 |
| | | ALAKA | ACTGCGCTAGCAAAAGCT | |

The expression construct may be designed to insert a sequence into a methylotrophic cell genome or to be transiently or stably expressed in an episomal construct. Constructs useful for integration into a methylotrophic cell minimally include a targeting sequence flanking an insertion sequence. The targeting sequence determines the locus sequence in the genome where the construct will be integrated. In some embodiments, the targeting sequence is a promoter (e.g. OLE1, AOX1, GAPDH, or DAS2 promoter) or another gene (e.g. PIF1). A targeting sequence may encompass the promoter when the construct inserts at the native locus of the promoter. A targeting sequence may include a nucleic acid sequence of from about 10 bp to about 10,000 bp (e.g., 10 bp-100 bp, e.g., 10 bp, 20 bp, 30 bp, 40 bp, 50 bp, 60 bp, 70 bp, 80 bp, 90 bp, 100 bp, e.g. 100 bp-1000 bp, e.g., 200 bp, 300 bp, 400 bp, 500 bp, 600 bp, 700 bp, 800 bp, 900 bp, 1000 bp, e.g., 1,000 bp-10,000 bp, e.g., 1,000 bp, 2,000 bp, 3,000 bp, 4,000 bp, 5,000 bp, 6,000 bp, 7,000 bp, 8,000 bp, 9,000 bp, 10,000 bp) that may enable efficient homologous recombination.

Heterologous proteins may be inserted into the genome of a methylotrophic cell at any suitable locus. Such loci include the native locus of the promoter employed or an alternative locus, such as the locus of a different promoter. Exemplary loci for use in the present invention include that of the OLE1, DAS2, AOX1, or GAPDH promoters or PIF1 (e.g., SEQ ID NO: 65).

Also provided herein are methods of preparing transgene expression constructs for expressing a heterologous protein comprising: (i) selecting a promoter that increases expression of one or more genes of the Mut pathway upon integration; or (ii) selecting a targeting sequence for guided recombination into a locus, wherein insertion of the heterologous protein into the locus increases expression of one or more genes of the Mut pathway; or (i) and (ii).

PIF1 Locus
SEQ ID NO: 65
TCACATTCTTTCACTCTACAAAATGACCAGAGTACGAAATATACGCATAC
ATTCGATTCAAGTTTTTTAAAGCCTTACATCGTATGTCTGGCAAAATCAG
AGAATGCCTCGTGAAAGAAAAAGACTGAATCCATTAACTTGCATGCCAAC
TCAATCCCGACTGTCAATCATTCATCCTTGCGTCTTTTGAACATCTATGC
TTCCACAAGTCAATTCTTGATTTAGTATACACATAACCAAATTTGGATCA
AGTTTGAAGTAAAACTTTAACTTCAGCTCCTTACATTTGCACTAAGATCT
CTGCTACTCTGGTCCCAAGTGAACCACCTTTTGGACCCTATTGACCGGAC
CTTAACTTGCCAAACCTAAACGCTTAATGCCTCAGACGTTTTAATGCCTC
TCAACACCTCCAAGGTTGCTTTCTTGAGCATGCCTACTAGGAACTTTAAC
GAACTGTGGGGTTGCAGACAGTTTCAGGCGTGTCCCGACCAATATGGCCT
ACTAGACTCTCTGAAAAATCACAGTTTTCCAGTAGTTCCGATCAAATTAC
CATCGAAATGGTCCCATAAACGGACATTTGACATCCGTTCCTGAATTATA

Alternatively, the heterologous protein may be expressed from an expression construct that is not integrated in the genome of the methylotrophic cell.

Sequences for other possible elements of expression constructs are known in the art. For example, transcription terminator sequence, origin of replication, multi-cloning site, and an antibiotic resistance marker sequences are known.

### Untranslated Regions (UTRs) and Kozak Sequences

The methylotrophic cells and expression constructs of the present disclosure may encode a nucleic acid comprising one or more regions or sequences which act or function as an untranslated region (UTR). As their name implies, UTRs are transcribed but not translated. In mRNA, the 5′ UTR is located directly upstream (5′) from the start codon (the first codon of an mRNA transcript translated by a ribosome). The first nucleic acid in the start codon is designated as +1 and nucleic acids located upstream are as designated as −1, −2, −3 and so on, while nucleic acids located downstream of this first nucleic acid are designated as +2, +3, +4 and so on. In some embodiments of the present disclosure, at least one 5′ untranslated region (UTR) is located upstream from the start codon of the nucleic acid encoding a heterologous protein of interest.

5′UTRs may harbor Kozak sequences, which are commonly involved in translation initiation. While Kozak sequences are known to broadly affect translation efficiency, study of the effect of a consensus Kozak sequence in Pichia has been heretofore limited. This disclosure is premised in part on the discovery of promoters (including but not limited to the DAS2, OLE1, AOX1, and SIT1 promoters) causing increased titers of downstream coding sequences, in part, because the promoters comprise enhanced Kozak sequences, leading to high translation efficiency.

Exemplary Kozak sequences include the Kozak sequence located in the 5′ UTR of nucleic acids encoding AOX1, DAS2, OLE1 and SIT1. For example, the Kozak sequence starting at the −4 position relative to the translation start site of the nucleic acid encoding the heterologous protein of interest may be AAAAATG. CACAATG, or AACGATG.

In some embodiments, the Kozak sequence is a native Kozak sequence (i.e., a Kozak sequence found in nature associated with the heterologous protein of interest). In some embodiments, the Kozak sequence is a heterologous Kozak sequence (i.e., a Kozak sequence found in nature not associated with the heterologous protein of interest). In some embodiments, the Kozak sequence is a synthetic Kozak sequence, which does not occur in nature. Synthetic Kozak sequences include sequences that have been mutated to improve their properties (e.g., which increase expression of a heterologous protein of interest). Synthetic Kozak sequences may also include nucleic acid analogues and chemically modified nucleic acids.

In some embodiments, the Kozak sequences of the present disclosure may begin at the −3 position relative to the translation start site of the nucleic acid encoding the heterologous protein of interest. In some embodiments, the Kozak sequence of the present disclosure comprises an adenine (A) at the −3 position and an adenine (A) at the −1 position relative to the translation start site of the nucleic acid encoding the heterologous protein of interest. In some embodiments, the Kozak sequence may comprise the sequence AN₁A starting at the −3 position relative to the translation start site of the nucleic acid encoding the heterologous protein of interest. The N₁ in the AN₁A sequence may be any nucleic acid. In some embodiments, the N₁ in AN₁A is adenine (A). In some embodiments, the N₁ in AN₁A is cytosine (C). In some embodiments, the N₁ in AN₁A is guanine (G). In some embodiments, the N₁ in AN₁A is thymine (T). In some embodiments, the Kozak sequence is AN₁AATGN₂C starting at the −3 position. The N₂ in the may be any nucleic acid. In some embodiments, N₂ is adenine (A). In some embodiments, N₂ is cytosine (C). In some embodiments, N₂ is guanine (G). In some embodiments, N₂ is thymine (T). In some embodiments, the Kozak sequence, starting at the −3 position relative to the translation start site, is A(A/C)(A/C), in which the −3 position is adenine (A), the −2 position is adenine (A) or cytosine (C) and the −1 position is either Adenine (A) or cytosine (C). In some embodiments, the Kozak sequence starting at the −3 position is A(A/C)(A/C)ATG.

Kozak sequences increase expression of a heterologous protein. In some embodiments, a Kozak sequence may increase expression of a heterologous protein at least 2-fold, at least 3-fold, at least 4-fold, at least 5-fold, at least 10-fold, at least 50-fold, at least 100-fold, at least 1000-fold compared to a control under similar or substantially similar conditions. In some embodiments, the control is the level of heterologous protein expression using a Kozak sequence that does not have an adenine (A) at the −1 position relative to the translation start site. In some embodiments, the control is the level of heterologous protein expression using a Kozak sequence that does not have an adenine (A) at the −3 position relative to the translation start site. In some embodiments, the control is the level of heterologous protein expression using a Kozak sequence that does not have an adenine (A) at the −3 position or the −1 position relative to the translation start site.

Secondary Structures in mRNA

Complementary base pairing in mRNA often gives rise to secondary structures. As used herein, secondary structures in mRNA include stem-loops (hairpins). Complementary base pairing in mRNA form the stem portion of a hairpin, while unpaired bases can form loops in the mRNA. Additional mRNA secondary structures include pseudoknots (see e.g., Staple et al., PLoS Biol. 3(6):e213, 2005). Algorithms known in the art may be used to predict mRNA secondary structure (see e.g., Matthews et al., Cold Spring Harb Perspect Biol. 2(12):a003665, 2010).

Free energy minimization can also be used to predict RNA secondary structure. For example, the stability of resulting helices (regions with base pairing) and loop regions often promote the formation of stem-loops in RNA. Parameters that affect the stability of double helix formation include the length of the double helix, the number of mismatches, the length of unpaired regions, the number of unpaired regions, the type of bases in the paired region and base stacking interactions. For example, guanine and cytosine can form three hydrogen bonds, while adenine and uracil form two hydrogen bonds. Thus, guanine-cytosine pairings are more stable than adenine-uracil pairings. Loop formation may be limited by steric hindrance, while base-stacking interactions stabilize loops. As an example, tetraloops (loops of four base pairs) often cap RNA hairpins and common tetraloop sequences include UNCG (N=A, C, G, or U).

In some embodiments, the secondary structure is any structure as predicted by likelihood of pairing and/or low free energy. In some embodiments, the secondary structure is a hairpin loop. In some embodiments, the secondary structure is a duplex, a single-stranded region, a hairpin, a bulge, or an internal loops.

Secondary structures may interfere with translation (e.g., block translation initiation and prevent translation elongation). For example, secondary structures in the 5′ UTR may disrupt binding of the ribosome and/or formation of the ribosomal initiation complex on mRNA. Secondary structures downstream of the translation start site, may prevent translation elongation. In some embodiments, a secondary structure in mRNA decreases total expression of a heterologous protein of interest relative to an mRNA without the secondary structure (e.g., reduces total expression by at least 2-fold, at least 3-fold, at least four-fold, at least 5-fold, at least 10-fold, at least 100-fold, at least 1000-fold). In some embodiments, a secondary structure in mRNA, e.g., a hairpin loop or any other structure as predicted by likelihood of pairing and/or low free energy, decreases expression of a full length version of a heterologous protein of interest (e.g., reduces expression by at least 2-fold, at least 3-fold, at least four-fold, at least 5-fold, at least 10-fold, at least 100-fold, at least 1000-fold). In some embodiments, a secondary structure in mRNA increases expression (e.g., by at least 2-fold, at least 3-fold, at least four-fold, at least 5-fold, at least 10-fold, at least 100-fold, at least 1000-fold) of at least one truncated form of a heterologous protein of interest.

Codon optimization, using one or more synonymous mutations that do not alter the amino acid sequence, may be used to mitigate the formation of secondary structures in mRNA encoding a heterologous protein of interest. In some embodiments, codon optimization reduces the number of complementary base pairs in the mRNA. In some embodiments, codon optimization of an mRNA encoding a heterologous protein of interest increases expression of the heterologous protein by at least 10%, at least 20%, at least 30%, at least 40%, at least 50%, at least 60%, at least 70%, at least 80%, at least 90% or at least 100% compared to a control mRNA sequence that encodes the heterologous protein but is not codon optimized.

### Methods of Heterologous Protein Production

### Integration of Expression Construct

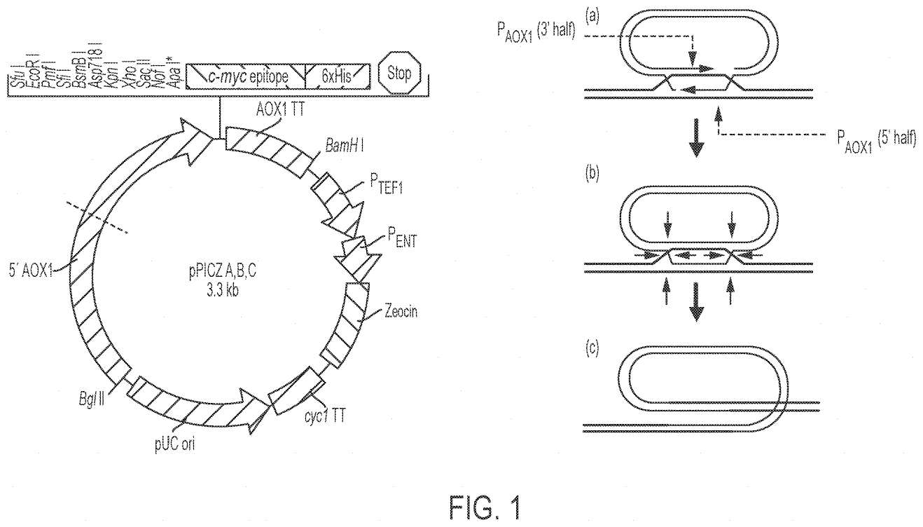

Heterologous protein production begins with the design of the expression construct carrying the gene of interest. Methods for introducing such constructs are known in the art. For example a construct may be designed for homologous recombination at a particular chromosomal locus in a methylotrophic cells, e.g., yeast. Once transformed (e.g. via electroporation, heat shock, lithium acetate), single or multi-copy strains are typically selected based on an antibiotic resistance gene (e.g., Zeocin (phleomycin Dl)). Higher-copy strains are generally achieved by iterative selection on increasing concentrations of antibiotic. The plasmid is directed to a specific locus by the target sequence on each end of the linearized cassette (FIG. 1).

### Fermentation

Methylotrophic cells, e.g., yeast, can be cultured via common methods known in the art such as in a shaker flask in an incubator at optimal growth temperatures (e.g., about 25° C.). Culture sizes can be scaled up so as to increase protein yield. First the cells are grown to a suitable cell density such that sufficient biomass is present. Cultures can be grown in media containing glucose or glycerol as the carbon source to promote efficient production of biomass. For example, cultures can be inoculated in buffered glycerol-containing media (BMGY, 4% v/v glycerol, 10 g/L yeast extract, 20 g/L peptone, 13.4 g/L yeast nitrogen base, 0.1 M potassium phosphate buffer pH 6.5) for about 24 hours. The glycerol concentration may vary from about 1% to about 5% (e.g. about 1%, 2%, 3%, 4%, or 5%). When the culture achieves a desired cell density (e.g., OD₆₀₀ 0.2-1.0) after about 24 hours, the medium is switched to a medium containing a different carbon source (e.g., methanol), which activates expression of genes under control of an inducible promoter, such as OLE1, DAS2, and AOX1. In some embodiments, a constitutively active promoter such as GAPDH can be used. For example, the medium is switched to buffered methanol-containing media (BMMY, 1.5% (v/v) methanol, 10 g/L yeast extract, 20 g/L peptone, 13.4 g/L yeast nitrogen base, 0.1 M potassium phosphate buffer pH 6.5) and the culture is grown for about 24 hours. The methanol concentration may vary from about 0.01% to about 10% (e.g. 0.01%-0.1%, e.g. 0.01%, 0.02%, 0.03%, 0.04%, 0.05%, 0.06%, 0.07%, 0.08%, 0.09%, 0.1%, e.g., 0.1%-1%, e.g. 0.2%, 0.3%, 0.4%, 0.5%, 0.6%, 0.7%, 0.8%, 0.9%, 1.0%, e.g., 1%-10%, e.g. 2%, 3%, 4%, 5%, 6%, 7%, 8%, 9%, 10%). After about 24 hours after induction with BMMY, the culture may be supplemented with additional 1.5% (v/v) methanol carbon source. The methanol supplement concentration may vary from about 0.01% to about 10% (e.g. 0.01%-0.1%, e.g. 0.01%, 0.02%, 0.03%, 0.04%, 0.05%, 0.06%, 0.07%, 0.08%, 0.09%, 0.1%, e.g., 0.1%-1%, e.g. 0.2%, 0.3%, 0.4%, 0.5%, 0.6%, 0.7%, 0.8%, 0.9%, 1.0%, e.g., 1%-10%, e.g. 2%, 3%, 4%, 5%, 6%, 7%, 8%, 9%, 10%). The culture may be grown for about an additional 24 hours, after which the cells may be harvested. Other modes of fermentation are known, e.g., chemostat and perfusion. The heterologous protein is secreted by the cells and can be purified using known methods. Protein expression levels, purity, and identity can be assayed e.g., with SDS-PAGE analysis, ELISA, and mass spectrometry.

### EXAMPLES

### Example 1. Identifying Genes Expressed in Glycerol and Methanol Conditions

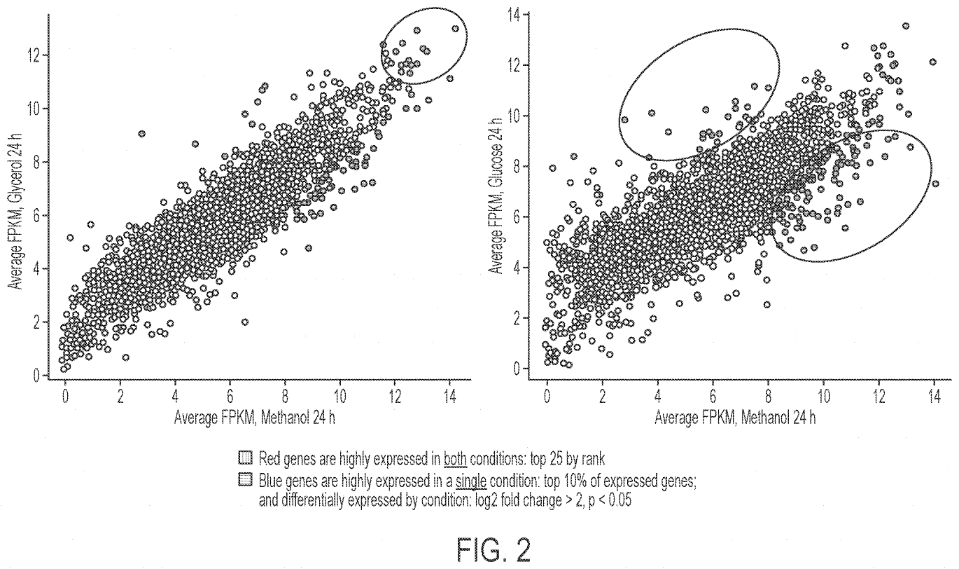

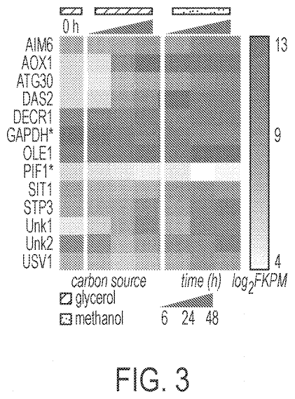

Gene expression profiles of K. phaffii were analyzed using RNA-Seq under either glycerol or glucose conditions first, and then methanol growth conditions (FIG. 2). Genes labeled in red were highly expressed under both conditions, while genes labeled in blue were differentially expressed and highly expressed under a single condition. From these data, promoters were tested for differential expression. P. pastoris was grown for 24 hours on glycerol, followed by 48 hours on either glycerol or methanol. Gene expression data are shown in FIG. 3.

### Example 2. Engineering a DNA Integration Plasmid

Heterologous protein production began with the design of the integration cassette carrying the gene of interest. Once transformed with the purified, linearized plasmid, single or multi-copy strains were selected on Zeocin. Higher-copy strains were achieved by iterative selection on increasing concentrations of Zeocin. Promoter sequences were selected by taking the 5′ UTR intergenic region, up to 1000 bp. Each promoter was either used as both the promoter sequence and integration locus, or preceded by the AOX1 or GAPDH promoter sequence for integration in the AOX1 or GAPDH locus. Each promoter was used to express human growth hormone (hGH) fused to the 5′ MFα (α mating factor) signal sequence. Promoter-ahGH sequences were synthesized by GeneArt (Invitrogen) and cloned in either the pPICZA (AOX1 locus) or pGAPZA (GAPDH locus) vectors. Two additional vectors were created for the AOX1 and DAS2 promoters using the PIF1 gene sequence as the locus, which flanks the GAPDH locus, to evaluate the presence of promoter contamination by the GAPDH promoter on the AOX1 or DAS2 promoters.

### Example 3. Detecting Protein Secretion Titers

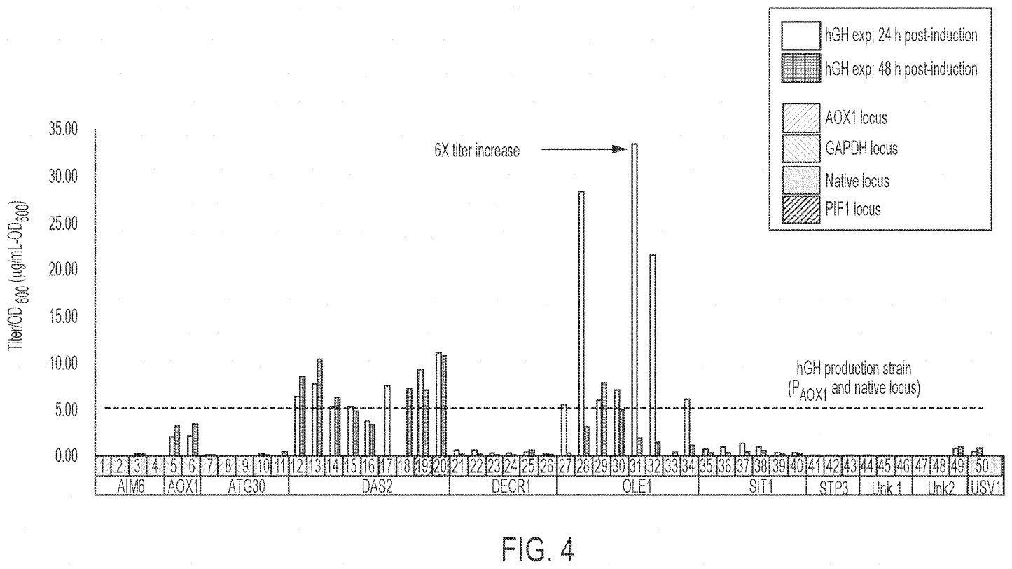

Vectors were linearized in the integration locus sequence and transformed by electroporation into wild-type P. pastoris by Blue Sky Biosciences (Worcester, Mass.). Clonal stocks were screened by immunoblot, and the top 1 or 2 clones per construct were evaluated in triplicate in 3-mL deep-well cultivation plates. Supernatant hGH titers were quantified by ELISA (FIG. 4).

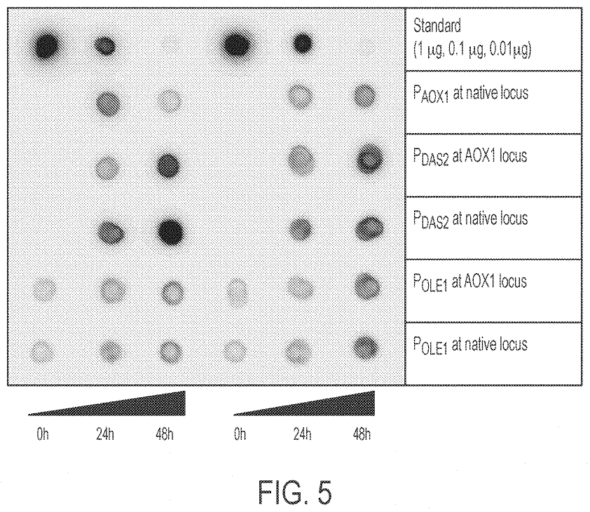

The results indicated that the promoter, and not the locus, dominated the phenotype, as the same promoter at various loci all produced comparable hGH titers. Compared to the benchmark hGH production strain (AOX1 at native locus), both the DAS2 and OLE1 promoters showed comparable or improved titers. A qualitative immunoblot (FIG. 5) was performed. DAS2 outperformed the benchmark at both scales, while OLE1 showed comparable results.

### Example 4. Identification of native secretion signal sequences

Native secretion signal sequences were identified by culturing K. phaffii cells and analyzing secreted proteins. Cultures were inoculated at 25° C. in buffered glycerol-containing media (BMGY, 4% (v/v) glycerol, 10 g/L yeast extract, 20 g/L peptone, 13.4 g/L yeast nitrogen base, 0.1 M potassium phosphate buffer pH 6.5) and grown for 24 hours during a biomass accumulation phase. Protein induction was achieved by switching the media to buffered methanol-containing media (BMMY, 1.5% (v/v) methanol, 10 g/L yeast extract, 20 g/L peptone, 13.4 g/L yeast nitrogen base, 0.1 M potassium phosphate buffer pH 6.5) and cultures were grown for 24 hours. Next, cultures were supplemented with 1.5% (v/v) methanol and grown for an additional 24 hours. 48 hours after induction, the cultures were harvested.

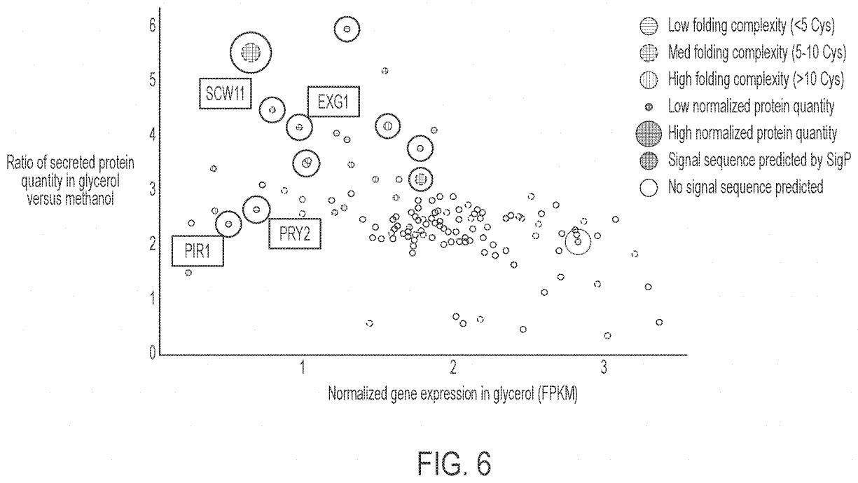

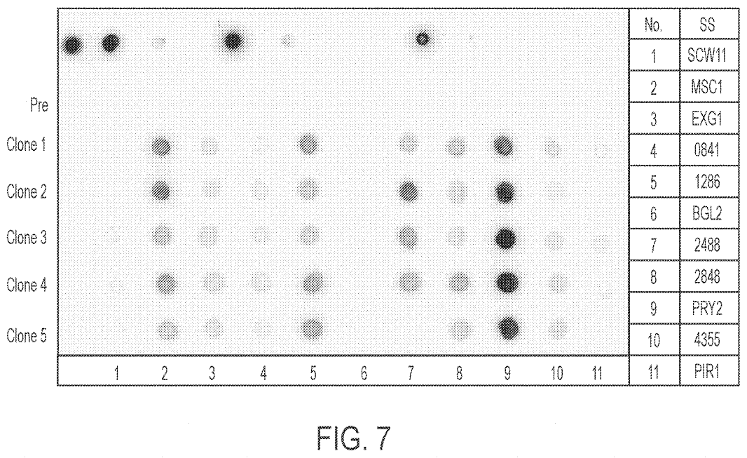

Proteins secreted during fermentation were analyzed by SDS-PAGE and LC-MS. These data were compared with quantification of mRNA transcripts (FIG. 6) so that efficient secretion signals could be identified. An immunoblot experiment was performed as in Example 3 to quantify expression of 11 candidate secretion signals, with PRY1 showing enhanced expression (FIG. 7).

### Example 5. Characterization of the DAS2 and AOX1 Promoters

This Example examined the effect of DAS2 and AOX1 promoters on expression of the human growth hormone (hGH) and also characterized the effect of these promoters on expression of endogenous methanol utilization pathway (Mut) genes. In particular, hGH cassettes carrying the DAS2 or AOX1 promoter were integrated into various loci and tested in P.pastoris. The results demonstrate that altered Mut pathway expression may enhance hGH productivity.

### Materials and Methods

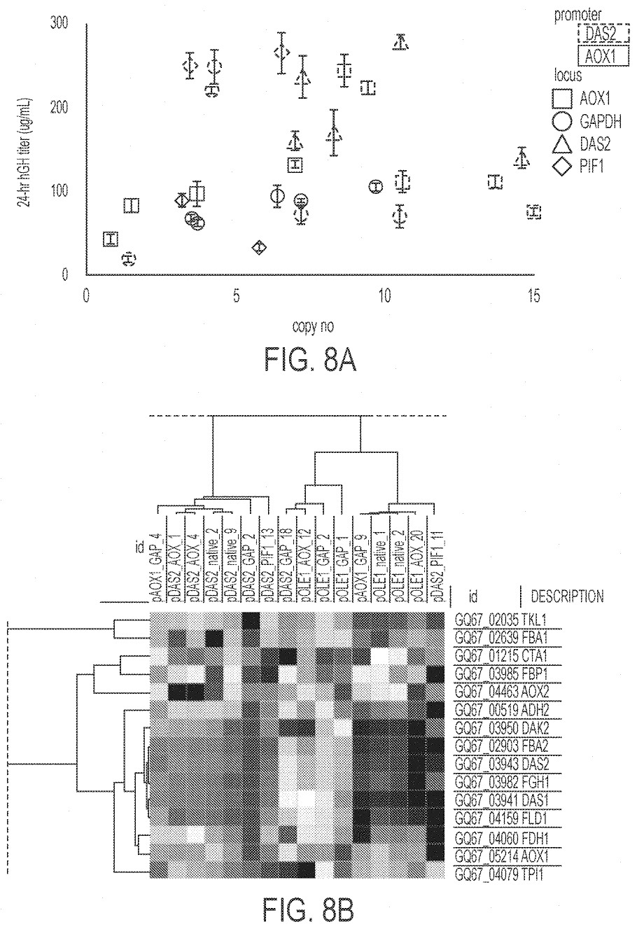

hGH protein titer was measured at 24 hr post-induction as a function of cassette copy number for strains in which hGH transgene expression is driven by a DAS2 promoter (referred to as PDAS2 or DAS2 strains) and for strains in which hGH transgene expression is driven by the AOX1 promoter (referred to as P_{AOX1} or AOX1 strains) at various loci (FIG. 8A). A heatmap was generated to compare expression of methanol utilization pathway (Mut) genes across high-producing strains (FIG. 8B).

### Results

Added benefits of upregulation of the DAS2 and AOX1 genes were surprisingly found: increased levels of transgene expression were detected when using these promoters and loci beyond what was expected for the level of transgene transcript observed in these strains via RNAseq.

As shown in FIG. 8B, these results were likely due to concomitant upregulation of the methanol utilization (Mut) pathway when using these promoters and loci. In the case of DAS2, use of this promoter at any of the tested loci leads to upregulation of the Mut pathway (FIG. 8B), which also was not expected. DAS2 strains display upregulated Mut, particularly of DAS1 and DAS2 strains, relative to other high-producers (FIG. 8B). Further, this upregulation can contribute to more than 2× protein titers in the case of the DAS2-based expression approach. As demonstrated in FIG. 8A, DAS2 strains produce greater than 2× the hGH protein titers compared to AOX1 strains with similar transgene copy number.

These results suggest that altered Mut pathway expression may further enhance hGH productivity.

### Example 6. Identification of a Consensus Kozak Sequence

This Example analysed 5′ UTR sequences from various gene promoters from P. pastoris to determine a consensus Kozak sequence and compared the translation efficiencies of each 5′UTR to direct heterologous expression of hGH.

### Materials and Methods

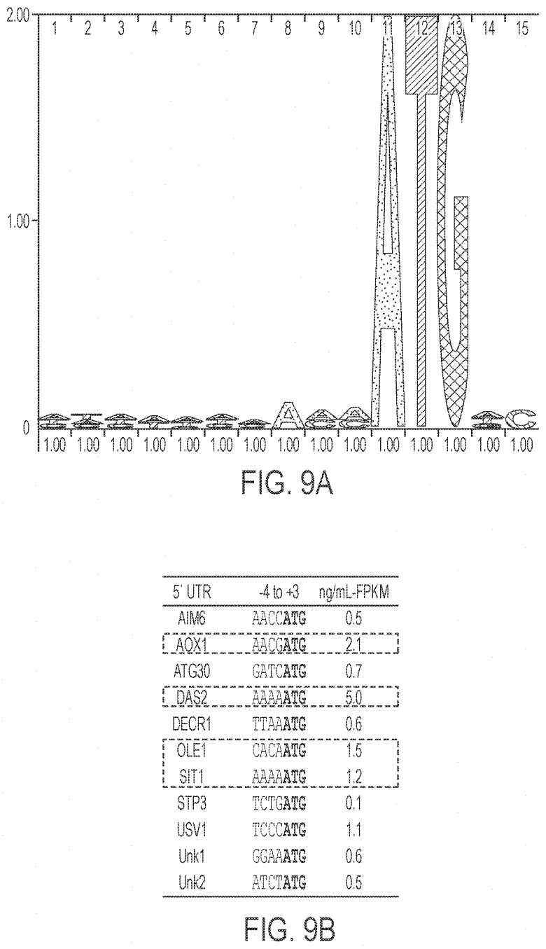

A HMM Logo of Kozak sequences across all P. pastoris genes was generated by Skylign given input aligned sequences (FIG. 9A). The height of each nucleotide in FIG. 9A is the information content without background (positive information content values only). Translation efficiency for each promoter/5′UTR used to direct heterologous gene expression was measured as ng/mL hGH in culture medium 24-hr post-induction per normalized hGH expression, as fragments per kilobase-pair per million reads (FPKM) (FIG. 9B).

### Results

A preferential Kozak sequence of ANAATGNC was discovered. As shown in FIG. 9A, there is a preference of A(A/C)(A/C)ATG across all P. pastoris genes. A 40% threshold for the most prominent nucleotide was used in this sequence and it was also required that the second-most prominent nucleotide occur 25% of the time or less. The 5′ UTR sequence included as part of the DAS2, OLE1, and SIT1 promoter sequences in the promoter studies also matches this consensus (FIG. 9B) and DAS2 and OLE1 were unexpectedly productive promoters. The combination of beneficial Mut pathway upregulation and optimal Kozak sequence correlates with the high productivity seen when the DAS2 promoter is used to express heterologous proteins, especially at its native locus.

### Example 7. Characterization of the Effect of Codon Optimization on Expression of Full Length VP8* and on Expression of N-Terminally Truncated VP8* Variants

This Example analyzed whether use of codon optimization to mitigate mRNA hairpin formation for VP8* would affect expression of full length VP8* and N-terminally truncated VP8* variants.

### Materials and Methods

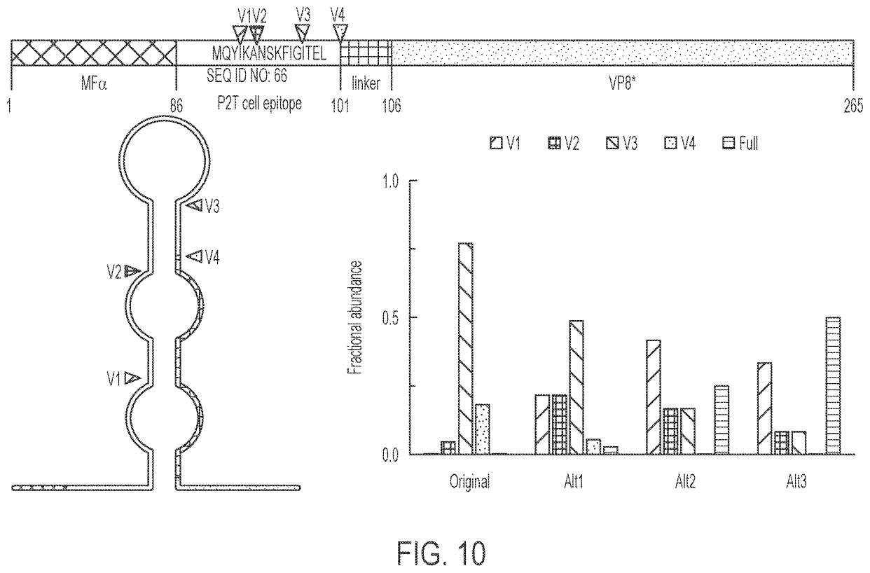

The desired full length VP8* protein consists of residues 86 through 265, directly following the alpha mating factor (uMF) signal sequence (FIG. 10, top diagram). V1, V2, V3 and V4 represent N-terminal VP8* variants (N-terminally truncated proteins), which correlate with the existence of the hairpin (shown in FIG. 10, bottom left). This hairpin was systematically mitigated using codon optimization that does not change the primary protein sequence.

### Results

As shown in FIG. 10, the predicted mRNA secondary structure of a protein can be systematically mitigated, significantly increasing the proportion of full-length secreted protein in cases where N-terminal truncations are observed. In particular, each alternative codon optimization (Alt1-5 codon changes, Alt2-6 codon changes, Alt3-7 codon changes) led to increased expression of the full length protein (FIG. 10 bar graph on the lower right). mRNA secondary structure mitigation has hitherto not been used as a lever for enhanced product quality, and its effect on quality has not been described. Unproductive mRNA structures, including hairpins, loops and other larger tertiary forms, may also be implicated in site-specific protein post-translational modifications, including glycosylation.

Thus, through the combination of promoter/locus selection (such as DAS2), an optimal Kozak sequence (ANA), and an mRNA sequence which lacks predicted, strong secondary structure, transgene cassette design can enable rapid and robust strain engineering for heterologous protein expression.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. An expression construct comprising an OLE1 promoter operably linked to a nucleic acid encoding a polypeptide comprising a signal sequence and a heterologous protein.

2. The expression construct of claim 1, wherein the signal sequence is identical to the signal sequence of a naturally occurring yeast protein.

3. The expression construct of claim 1 or 2, wherein the signal sequence is the signal sequence of SCW11, MSC1, EXG1, 0841, 1286, BGL2, 2488, 2848, PRY2, 4355, or PIR1.

4. The expression construct of any one of claims 1 to 3, wherein the OLE1 promoter has at least 95% homology with SEQ ID NO: 1 or a fragment thereof.

5. The expression construct of claim 4, wherein the OLE1 promoter has the sequence SEQ ID NO: 1.

6. The expression construct of any one of claims 1 to 5, wherein the expression construct is a plasmid or viral vector.

7. The expression construct of claim 6, wherein the plasmid is an episomal plasmid or an integrative plasmid.

8. The expression construct of any one of claims 1 to 7, wherein the expression construct is linearized.

9. The expression construct of any one of claims 1 to 8, wherein the heterologous protein is selected from the group consisting of enzymes, hormones, antibodies or antigen binding fragments thereof, vaccine components, blood factors, thrombolytic agents, cytokines, receptors, and fusion proteins.

10. A methylotrophic cell expressing a heterologous protein, wherein the expression is under the control of an OLE1 promoter.

11. The methylotrophic cell of claim 10, wherein the cell has been transformed by the expression construct of any of claims 1-9.

12. The methylotrophic cell of claim 10 or 11, wherein the OLE1 promoter is located at the OLE1, AOX1, GAPDH, DAS2, or PIF1 locus.

13. The methylotrophic cell of any one of claims 10 to 12, wherein the methylotrophic cell is a yeast cell.

14. The methylotrophic cell of claim 13, wherein the yeast cell is a Pichia pastoris cell.

15. The methylotrophic cell of claim 14, wherein the Pichia pastoris cell is a Komagataella phaffii or Komagataella pastoris cell.

16. The methylotrophic cell of claim 15, wherein the Komagataella phaffii cell is a Komagataella phaffii Y-11430, Y-7556, YB-4290, Y-12729, Y-17741, Y-48123, Y-48124, YB-378, YB-4289, GS115, KM71H, SMD1168, SMD11681-1, or X-33 cell.

17. The methylotrophic cell of any one of claims 10 to 16, wherein the OLE1 promoter has at least 95% homology with SEQ ID NO: 1 or a fragment thereof.

18. The methylotrophic cell of claim 17, wherein the OLE1 promoter has the sequence SEQ ID NO: 1.

19. The methylotrophic cell of any one of claims 10 to 18, wherein the heterologous protein is selected from the group consisting of enzymes, hormones, antibodies or antigen binding fragments thereof, vaccine components, blood factors, thrombolytic agents, cytokines, receptors, and fusion proteins.

20. The methylotrophic cell of any one of claims 10 to 19, further comprising a signal sequence fused to the heterologous protein.

21. The methylotrophic cell of claim 20, wherein the signal sequence is the signal sequence of SCW11, MSC1, EXG1, 0841, 1286, BGL2, 2488, 2848, PRY2, 4355, or PIR1.

22. An expression construct comprising a DAS2 promoter operably linked to a nucleic acid encoding a polypeptide comprising a signal sequence and a heterologous protein and a targeting sequence for integration in a methylotrophic cell at a non-native locus.

23. The expression construct of claim 22, wherein the signal sequence is identical to the signal sequence of a naturally occurring yeast protein.

24. The expression construct of claim 22 or 23, wherein the signal sequence is the signal sequence of SCW11, MSC1, EXG1, 0841, 1286, BGL2, 2488, 2848, PRY2, 4355, or PIR1.

25. The expression construct of any one of claims 22-24, wherein the DAS2 promoter has at least 95% homology with SEQ ID NO: 2 or a fragment thereof.

26. The expression construct of claim 25, wherein the DAS2 promoter has the sequence SEQ ID NO: 2.

27. The expression construct of any one of claims 22-26, wherein the expression construct is a plasmid or viral vector.

28. The expression construct of claim 27, wherein the plasmid is an episomal plasmid or an integrative plasmid.

29. The expression construct of any one of claims 22 to 28, wherein the expression construct is linearized.

30. The expression construct of any one of claims 22-29, wherein the heterologous protein is selected from the group consisting of enzymes, hormones, antibodies or antigen binding fragments thereof, vaccine components, blood factors, thrombolytic agents, cytokines, receptors, and fusion proteins.

31. The expression construct of any one of claims 22-30, wherein the expression construct further comprises a Kozak sequence beginning at the −3 position relative to the translation start site of the nucleic acid encoding the polypeptide.

32. The expression construct of claim 31, wherein the Kozak sequence comprises:
(i) the sequence ANAATGNC, wherein N comprises A, T, G, or C; or
(ii) the sequence AMMATG, wherein M comprises A or C.

33. The expression construct of any one of claims 22-32, wherein a mRNA secondary structure of the nucleic acid encoding a polypeptide has been reduced or eliminated relative to the endogenous nucleic acid encoding the polypeptide.

34. The expression construct of claim 33, wherein the mRNA secondary structure is selected from a hairpin loop, a duplex, a single-stranded region, a hairpin, a bulge, an internal loop, or any other structure as predicted by likelihood of pairing and/or low free energy.

35. A methylotrophic cell expressing a heterologous protein, wherein the expression is under the control of a DAS2 promoter integrated at a non-native locus.

36. The methylotrophic cell of claim 35, wherein the non-native locus is an OLE1, AOX1, GAPDH, or PIF1 locus.

37. The methylotrophic cell of claim 35 or 36, wherein the methylotrophic cell is a yeast cell.

38. The methylotrophic cell of claim 37, wherein the yeast cell is a Pichia pastoris cell.

39. The methylotrophic cell of claim 38, wherein the Pichia pastoris cell is a Komagataella phaffii or Komagataella pastoris cell.

40. The methylotrophic cell of claim 39, wherein the Komagataella phaffii cell is a Komagataella phaffii Y-11430, Y-7556, YB-4290, Y-12729, Y-17741, Y-48123, Y-48124, YB-378, YB-4289, GS115, KM71H, SMD1168, SMD1168H, or X-33 cell.

41. The methylotrophic cell of any one of claims 35 to 40, wherein the DAS2 promoter has at least 95% homology with SEQ ID NO: 2.

42. The methylotrophic cell of claim 41, wherein the DAS2 promoter has the sequence SEQ ID NO: 2.

43. The methylotrophic cell of any one of claims 35 to 42, wherein the heterologous protein is selected from the group consisting of enzymes, hormones, antibodies or antigen binding fragments thereof, vaccine components, blood factors, thrombolytic agents, cytokines, receptors, and fusion proteins.

44. The methylotrophic cell of any one of claims 35 to 43, further comprising a signal sequence fused to the heterologous protein.

45. The methylotrophic cell of claim 44, wherein the signal sequence is the signal sequence of SCW11, MSC1, EXG1, 0841, 1286, BCL2, 2488, 2848, PRY2, 4355, or PIR1.

46. The methylotrophic cell of any one of claims 35-45, wherein the mRNA encoding the heterologous protein comprises a Kozak sequence beginning at the −3 position relative to the translation start site.

47. The methylotrophic cell of claim 46, wherein the Kozak sequence comprises:
(i) the sequence ANAATGNC, wherein N comprises A, T, G, or C; or
(ii) the sequence AMMATG, wherein M comprises A or C.

48. The methylotrophic cell of any one of claims 35-47, wherein a mRNA secondary structure of the mRNA encoding the heterologous protein has been reduced or eliminated relative to the endogenous mRNA encoding the heterologous protein.

49. The methylotrophic cell of claim 48, wherein the mRNA secondary structure is selected from a hairpin loop, a duplex, a single-stranded region, a hairpin, a bulge, an internal loop, or any other structure as predicted by likelihood of pairing and/or low free energy.

50. An expression construct comprising an AOX1 promoter operably linked to a nucleic acid encoding a polypeptide comprising a signal sequence and a heterologous protein, the construct further comprising a targeting sequence for integration in a methylotrophic cell at a PIF1, OLE1, or DAS2 locus.

51. The expression construct of claim 50, wherein the signal sequence is identical to the signal sequence of a naturally occurring yeast protein.

52. The expression construct of claim 50-51, wherein the signal sequence is the signal sequence of SCW11, MSC1, EXG1, 0841, 1286, BGL2, 2488, 2848, PRY2, 4355, or PIR1.

53. The expression construct of any one of claims 50-52, wherein the AOX1 promoter has at least 95% homology with SEQ ID NO: 3 or a fragment thereof.

54. The expression construct of claim 53, wherein the AOX1 promoter has the sequence SEQ ID NO: 3.

55. The expression construct of any one of claims 50-54, wherein the expression construct is a plasmid or viral vector.

56. The expression construct of claim 55, wherein the plasmid is an episomal plasmid or an integrative plasmid.

57. The expression construct of any one of claims 50-56, wherein the expression construct is linearized.

58. The expression construct of any one of claims 50-57, wherein the heterologous protein is selected from the group consisting of enzymes, hormones, antibodies or antigen binding fragments thereof, vaccine components, blood factors, thrombolytic agents, cytokines, receptors, and fusion proteins.

59. The expression construct of any one of claims 50-58, wherein the expression construct further comprises a Kozak sequence beginning at the −3 position relative to the translation start site of the nucleic acid encoding the polypeptide.

60. The expression construct of claim 59, wherein the Kozak sequence comprises:
(i) the sequence ANAATGNC, wherein N comprises A, T, G, or C; or
(ii) the sequence AMMATG, wherein M comprises A or C.

61. The expression construct of any one of claims 50-60, wherein a mRNA secondary structure of the nucleic acid encoding a polypeptide has been reduced or eliminated relative to the endogenous nucleic acid encoding the polypeptide.

62. The expression construct of claim 61, wherein the mRNA secondary structure is selected from a hairpin loop, a duplex, a single-stranded region, a hairpin, a bulge, an internal loop, or any other structure as predicted by likelihood of pairing and/or low free energy.

63. A methylotrophic cell expressing a heterologous protein, wherein the expression is under the control of an AOX1 promoter integrated at a PIF1, OLE1, or DAS2 locus.

64. The methylotrophic cell of claim 63, wherein the methylotrophic cell is a yeast cell.

65. The methylotrophic cell of claim 64, wherein the yeast cell is a Pichia pastoris cell.

66. The methylotrophic cell of claim 65, wherein the Pichia pastoris cell is a Komagataella phaffii or Komagataella pastoris cell.

67. The methylotrophic cell of claim 66, wherein the Komagataella phaffii cell is a Komagataella phaffii
Y-11430, Y-7556, YB-4290, Y-12729, Y-17741, Y-48123, Y-48124, YB-378, YB-4289, GS115, KM71H, SMD1168, SMD1168H, or X-33 cell.

68. The methylotrophic cell of any one of claims 63-67, wherein the AOX1 promoter has at least 95% homology with SEQ ID NO: 3.

69. The methylotrophic cell of claim 68, wherein the AOX1 has the sequence SEQ ID NO: 3.

70. The methylotrophic cell of any one of claims 63-69, wherein the heterologous protein is selected from the group consisting of enzymes, hormones, antibodies or antigen binding fragments thereof, vaccine components, blood factors, thrombolytic agents, cytokines, receptors, and fusion proteins.

71. The methylotrophic cell of any one of claims 63-70, further comprising a signal sequence fused to the heterologous protein.

72. The methylotrophic cell of claim 71, wherein the signal sequence is the signal sequence of SCW11, MSC1, EXG1, 0841, 1286, BCL2, 2488, 2848, PRY2, 4355, or PIR1.

73. The methylotrophic cell of any one of claims 63-72, wherein the mRNA encoding the heterologous protein comprises a Kozak sequence beginning at the −3 position relative to the translation start site.

74. The methylotrophic cell of claim 73, wherein the Kozak sequence comprises:
(i) the sequence ANAATGNC, wherein N comprises A, T, Ci, or C; or
(ii) the sequence AMMATG, wherein M comprises A or C.

75. The methylotrophic cell of any one of claims 63-74, wherein a mRNA secondary structure of the mRNA encoding the heterologous protein has been reduced or eliminated relative to the endogenous mRNA encoding the heterologous protein.

76. The methylotrophic cell of claim 75, wherein the mRNA secondary structure is selected from a hairpin loop, a duplex, a single-stranded region, a hairpin, a bulge, an internal loop, or any other structure as predicted by likelihood of pairing and/or low free energy.

77. An expression construct comprising a GAPDH promoter operably linked to a nucleic acid encoding a polypeptide comprising a signal sequence and a heterologous protein, the construct further comprising a targeting sequence for integration in a methylotrophic cell at an AOX1, PIF1, OLE1, or DAS2 locus.

78. The expression construct of claim 77, wherein the signal sequence is identical to the signal sequence of a naturally occurring yeast protein.

79. The expression construct of claim 77 or 78, wherein the signal sequence is the signal sequence of SCW11, MSC1, EXG1, 0841, 1286, BCL2, 2488, 2848, PRY2, 4355, or PIR1.

80. The expression construct of any one of claims 77-79, wherein the GAPDH promoter has at least 95% homology with SEQ ID NO: 4 or a fragment thereof.

81. The expression construct of claim 80, wherein the GAPDH promoter has the sequence SEQ ID NO: 4.

82. The expression construct of any one of claims 77-81, wherein the expression construct is a plasmid or viral vector.

83. The expression construct of claim 82, wherein the plasmid is an episomal plasmid or an integrative plasmid.

84. The expression construct of any one of claims 77-83, wherein the expression construct is linearized.

85. The expression construct of any one of claims 77-84, wherein the heterologous protein is selected from the group consisting of enzymes, hormones, antibodies or antigen binding fragments thereof, vaccine components, blood factors, thrombolytic agents, cytokines, receptors, and fusion proteins.

86. The expression construct of any one of claims 77-85, wherein the expression construct further comprises a Kozak sequence beginning at the −3 position relative to the translation start site of the nucleic acid encoding the polypeptide.

87. The expression construct of claim 86, wherein the Kozak sequence comprises:
(i) the sequence ANAATGNC, wherein N comprises A, T, G, or C; or
(ii) the sequence AMMATG, wherein M comprises A or C.

88. The expression construct of any one of claims 77-87, wherein a mRNA secondary structure of the nucleic acid encoding a polypeptide has been reduced or eliminated relative to the endogenous nucleic acid encoding the polypeptide.

89. The expression construct of claim 88, wherein the mRNA secondary structure is selected from a hairpin loop, a duplex, a single-stranded region, a hairpin, a bulge, an internal loop, or any other structure as predicted by likelihood of pairing and/or low free energy.

90. A methylotrophic cell expressing a heterologous protein, wherein the expression is under the control of a GAPDH promoter integrated at an AOX1, PIF1, OLE1, or DAS2 locus.

91. The methylotrophic cell of claim 90, wherein the methylotrophic cell is a yeast cell.

92. The methylotrophic cell of claim 91, wherein the yeast cell is a Pichia pastoris cell.

93. The methylotrophic cell of claim 92, wherein the Pichia pastoris cell is a Komagataella phaffii or Komagataella pastoris cell.

94. The methylotrophic cell of claim 93, wherein the Komagataella phaffii cell is a Komagataella phaffii
Y-11430, Y-7556, YB-4290, Y-12729, Y-17741, Y-48123, Y-48124, YB-378, YB-4289, GS115, KM71H, SMD1168, SMD1168H, or X-33 cell.

95. The methylotrophic cell of any one of claims 90-94, wherein the GAPDH promoter has at least 95% homology with SEQ ID NO: 4.

96. The methylotrophic cell of claim 95, wherein the GAPDH promoter has the sequence SEQ ID NO: 4.

97. The methylotrophic cell of any one of claims 90-96, wherein the heterologous protein is selected from the group consisting of enzymes, hormones, antibodies or antigen binding fragments thereof, vaccine components, blood factors, thrombolytic agents, cytokines, receptors, and fusion proteins.

98. The methylotrophic cell of any one of claims 90-97, further comprising a signal sequence fused to the heterologous protein.

99. The methylotrophic cell of claim 98, wherein the signal sequence is the signal sequence of SCW11, MSC1, EXG1, 0841, 1286, BCL2, 2488, 2848, PRY2, 4355, or PIR1.

100. The methylotrophic cell of any one of claims 90-99, wherein the mRNA encoding the heterologous protein comprises a Kozak sequence beginning at the −3 position relative to the translation start site.

101. The methylotrophic cell of claim 100, wherein the Kozak sequence comprises:
(i) the sequence ANAATGNC, wherein N comprises A, T, G, or C; or
(ii) the sequence AMMATG, wherein M comprises A or C.

102. The methylotrophic cell of any one of claims 90-101, wherein a mRNA secondary structure of the mRNA encoding the heterologous protein has been reduced or eliminated relative to the endogenous mRNA encoding the heterologous protein.

103. The methylotrophic cell of claim 102, wherein the mRNA secondary structure is selected from a hairpin loop, a duplex, a single-stranded region, a hairpin, a bulge, an internal loop, or any other structure as predicted by likelihood of pairing and/or low free energy.

104. An expression construct comprising a promoter operably linked to a nucleic acid encoding a polypeptide comprising a signal sequence and a heterologous protein, wherein the signal sequence is a signal sequence of KAR2, MSC1, TOS1, 2241, LHS1, TIF1, CTS1, or 5326.

105. The expression construct of claim 104, wherein the promoter in an OLE1, AOX1, DAS2, or GAPDH promoter.

106. The expression construct of any one of claims 104-105, wherein the expression construct is a plasmid or viral vector.

107. The expression construct of claim 106, wherein the plasmid is an episomal plasmid or an integrative plasmid.

108. The expression construct of any one of claims 104-107, wherein the expression construct is linearized.

109. The expression construct of any one of claims 104-108, wherein the heterologous protein is selected from the group consisting of enzymes, hormones, antibodies or antigen binding fragments thereof, vaccine components, blood factors, thrombolytic agents, cytokines, receptors, and fusion proteins.

110. The expression construct of any of claims 104-109, further comprising a targeting sequence for integration in a methylotrophic cell at an AOX1, PIF1, OLE1, GAPDH, or DAS2 locus.

111. A methylotrophic cell expressing a heterologous protein fused to a signal sequence, wherein the signal sequence is a signal sequence of KAR2, MSC1, TOS1, 2241, LHS1, TIF1, CTS1, or 5326.

112. The methylotrophic cell of claim 111, wherein the methylotrophic cell is a yeast cell.

113. The methylotrophic cell of claim 112, wherein the yeast cell is a Pichia pastoris cell.

114. The methylotrophic cell of claim 113, wherein the Pichia pastoris cell is a Komagataella phaffii or Komagataella pastoris cell.

115. The methylotrophic cell of claim 114, wherein the Komagataella phaffii cell is a Komagataella phaffii
Y-11430, Y-7556, YB-4290, Y-12729, Y-17741, Y-48123, Y-48124, YB-378, YB-4289, GS115, KM71H, SMD1168, SMD1168H, or X-33 cell.

116. The methylotrophic cell of any one of claims 111-115, wherein the expression is under the control of an OLE1, AOX1, DAS2, or GAPDH promoter.

117. The methylotrophic cell of any one of claims 111-116, wherein the heterologous protein is selected from the group consisting of enzymes, hormones, antibodies or antigen binding fragments thereof, vaccine components, blood factors, thrombolytic agents, cytokines, receptors, and fusion proteins.

118. The methylotrophic cell of any of claims 111-117, wherein the heterologous protein is integrated at an AOX1, PIF1, OLE1, GAPDH, or DAS2 locus.

119. A method of producing a heterologous protein with the methylotrophic cell of any one of claims 10 to 21, 35-45, 63-72, 90-99, and 111-118, the method comprising culturing the cell under conditions suitable to express the heterologous protein.

120. The method of claim 119, further comprising first culturing the cell with a first carbon source lacking methanol under conditions in which the heterologous protein is substantially not expressed, followed by switching the carbon source to a carbon source that includes methanol to express the heterologous protein.

121. The method of any one of claims 119-120, further comprising isolating the protein.

122. A methylotrophic cell expressing a heterologous protein under the control of a promoter, wherein:
(i) the promoter is an AOX1 promoter or a DAS2 promoter and/or the promoter is located at an AOX1 or DAS2 locus;
(ii) mRNA encoding the heterologous protein comprises a Kozak sequence beginning at the −3 position relative to the translation start site; and/or
(iii) a mRNA secondary structure of the mRNA encoding the heterologous protein has been reduced or eliminated relative to the endogenous mRNA encoding the heterologous protein.

123. The methylotrophic cell of claim 122, wherein the methylotrophic cell is a yeast cell.

124. The methylotrophic cell of claim 123, wherein the yeast cell is a Pichia pastoris cell.

125. The methylotrophic cell of claim 124, wherein the Pichia pastoris cell is a Komagataella phaffii or Komagataella pastoris cell.

126. The methylotrophic cell of claim 125, wherein the Komagataella phaffii cell is a Komagataella phaffii Y-11430, Y-7556, YB-4290, Y-12729, Y-17741, Y-48123, Y-48124, YB-378, YB-4289, GS115, KM71H, SMD1168, SMD1168H, or X-33 cell.

127. The methylotrophic cell of any one of claims 122-126, wherein the AOX1 promoter has at least 95% homology with SEQ ID NO: 3 or a fragment thereof.

128. The methylotrophic cell of claim 127, wherein the AOX1 promoter has the sequence SEQ ID NO: 3.

129. The methylotrophic cell of any one of claims 122-126, wherein the DAS2 promoter has at least 95% homology with SEQ ID NO: 2 or a fragment thereof.

130. The methylotrophic cell of claim 127, wherein the DAS2 promoter has the sequence SEQ ID NO: 2.

131. The methylotrophic cell of any one of claims 122-130, wherein the heterologous protein is selected from the group consisting of an enzyme, hormone, antibody or antigen-binding antibody fragments, vaccine component, blood factor, thrombolytic agent, cytokine, receptor, and fusion protein.

132. The methylotrophic cell of any one of claims 122-131, wherein the heterologous protein is fused to a signal sequence.

133. The methylotrophic cell of claim 132, wherein the signal sequence is the signal sequence of SCW11, MSC1, EXG1, 0841, 1286, BCL2, 2488, 2848, PRY2, 4355, or PIR1.

134. The methylotrophic cell of any one of claims 122-133, wherein the Kozak sequence comprises:
(i) the sequence ANAATGNC, wherein N comprises A, T, G, or C; or
(ii) the sequence AMMATG, wherein M comprises A or C.

135. The methylotrophic cell of any one of claims 122-134, wherein the mRNA secondary structure is selected from a hairpin loop, a duplex, a single-stranded region, a hairpin, a bulge, an internal loop, or any other structure as predicted by likelihood of pairing and/or low free energy.

136. An expression construct comprising a promoter operably linked to a nucleic acid encoding a polypeptide comprising a signal sequence and a heterologous protein, wherein:
(i) the promoter is an AOX1 or DAS2 promoter and/or the construct further comprises a targeting sequence for integration in a methylotrophic cell at an AOX1 or DAS2 locus;
(ii) the expression construct further comprises a Kozak sequence beginning at the −3 position relative to the translation start site of the nucleic acid encoding the polypeptide; and/or
(iii) a mRNA secondary structure of the nucleic acid encoding a polypeptide has been reduced or eliminated relative to the endogenous nucleic acid encoding the polypeptide.

137. The expression construct of claim 136, wherein the signal sequence is identical to the signal sequence of a naturally occurring yeast protein.

138. The expression construct of claim 136 or 137, wherein the signal sequence is the signal sequence of SCW11, MSC1, EXG1, 0841, 1286, BGL2, 2488, 2848, PRY2, 4355, or PIR1.

139. The expression construct of any one of claims 136 to 138, wherein the AOX1 promoter has at least 95% homology with SEQ ID NO: 3 or a fragment thereof.

140. The expression construct of claim 139, wherein the AOX1 promoter has the sequence SEQ ID NO: 3.

141. The expression construct of any one of claims 136 to 138, wherein the DAS2 promoter has at least 95% homology with SEQ ID NO: 2 or a fragment thereof.

142. The expression construct of claim 141, wherein the DAS2 promoter has the sequence SEQ ID NO: 2.

143. The expression construct of any one of claims 136 to 142, wherein the expression construct is a plasmid or viral vector.

144. The expression construct of claim 143, wherein the plasmid is an episomal plasmid or an integrative plasmid.

145. The expression construct of any one of claims 136 to 144, wherein the expression construct is linearized.

146. The expression construct of any one of claims 136 to 145, wherein the heterologous protein is selected from the group consisting of an enzyme, hormone, antibody or antigen-binding antibody fragment, vaccine component, blood factor, thrombolytic agent, cytokine, receptor, and fusion protein.

147. The expression construct of any one of claims 136 to 146, wherein the Kozak sequence comprises:
(i) the sequence ANAATGNC, wherein N comprises A, T, G, or C; or
(ii) the sequence AMMATG, wherein M comprises A or C.

148. The expression construct of any one of claims 136 to 147, wherein the mRNA secondary structure is selected from a hairpin loop, a duplex, a single-stranded region, a hairpin, a bulge, an internal loop, or any other structure as predicted by likelihood of pairing and/or low free energy.

149. A method for preparing a transgene expression construct for expressing a heterologous protein in Pichia comprising:
providing a nucleic acid encoding a heterologous protein; and
(i) selecting a promoter that increases expression of genes of the Mut pathway upon integration; or
(ii) selecting a targeting sequence for guided recombination into a locus, wherein insertion of the heterologous protein into the locus increases expression of genes of the Mut pathway; or
(i) and (ii).

150. The method of claim 149, further comprising selecting a Kozak sequence beginning at the −3 position relative to the translation start site of the nucleic acid encoding the heterologous protein.

151. The method of claim 149 or 150, further comprising reducing or eliminating a mRNA secondary structure of the nucleic acid encoding a polypeptide has been reduced or eliminated relative to the endogenous nucleic acid encoding the polypeptide.

152. The method of any of claims 149-151, wherein the nucleic acid further encodes a signal sequence.

153. The method of claim 152, wherein the signal sequence is identical to the signal sequence of a naturally occurring yeast protein.

154. The method of claim 152 or 153, wherein the signal sequence is the signal sequence of SCW11, MSC1, EXG1, 0841, 1286, BGL2, 2488, 2848, PRY2, 4355, or PIR1.

155. The method of any one of claims 149-154, wherein the promoter is DAS1, DAS2, AOX1, GAPDH, and ATG30.

156. The method of any one of claims 149-155, wherein the locus is DAS1, DAS2, AOX1, GAPDH, and ATG30.

157. The expression construct of any one of claims 149-156, wherein the heterologous protein is selected from the group consisting of enzymes, hormones, antibodies or antigen binding fragments thereof, vaccine components, blood factors, thrombolytic agents, cytokines, receptors, and fusion proteins.

158. The method of any one of claims 149-157, wherein the Kozak sequence comprises:
(i) the sequence ANAATGNC, wherein N comprises A, T, G, or C; or
(ii) the sequence AMMATG, wherein M comprises A or C.

159. The method of any one of claims 149-158, wherein the mRNA secondary structure is selected from a hairpin loop, a duplex, a single-stranded region, a hairpin, a bulge, an internal loop, or any other structure as predicted by likelihood of pairing and/or low free energy.
